(19) United States
(12) Patent Application Publication
(10) Pub. No.: US 2022/0251126 A1
(43) Pub. Date: Aug. 11, 2022

(54) ANTICANCER AGENTS

(71) Applicant: Yissum Research Development Company of the Hebrew University of Jerusalem Ltd., Jerusalem (IL)

(72) Inventors: Dan GIBSON, Jerusalem (IL); Amrita SARKAR, Jerusalem (IL)

(73) Assignee: Yissum Research Development Company of the Hebrew University of Jerusalem Ltd., Jerusalem (IL)

(21) Appl. No.: 17/597,878

(22) PCT Filed: Aug. 5, 2020
(86) PCT No.: PCT/IL2020/050857

§ 371 Date: Jan. 27, 2022

Related U.S. Application Data
(60) Provisional application No. 62/882,770, filed on Aug. 5, 2019.

(51) Int. Cl. C07F 15/00; A61K 47/55; A61K 47/54

(52) U.S. Cl. CPC C07F 15/0093; A61K 47/547; A61K 47/552

(57) ABSTRACT

The claimed subject matter provides for a method for conjugating an active material to a Pt complex.

### TECHNOLOGICAL FIELD

The technology subject of the present invention concerns novel Pt-based anticancer agents and multi-action Pt-based anticancer agents with bioactive ligands, methods of preparation thereof and uses thereof.

### BACKGROUND

Despite many advances in cancer treatment, square planar Pt^{II} complexes such as cisplatin, second generation carboplatin and third generation oxaliplatin (FIG. 1) are widely used in the clinic to treat several forms of cancer, alone or in combination with other drugs. The agents are administered intravenously and after entering the cancer cell, the cis-[Pt(Am)₂]²⁺ binds to two adjacent guanines on the same strand of the DNA, distorting its structure and triggering cellular responses that result in apoptosis.

Pt drugs suffer from two major problems: the first having to do with the ability of the tumors to acquire resistance to these drugs and the second relating to dose-limiting side effects that are known to be associated with the chemotherapy. In order to overcome resistance to a given drug and diminish side effects, clinicians have been treating patients with a combination of drugs that have different cellular targets and different modes of action. One of the popular strategies is to use inert, octahedral multi-action Pt^{IV} complexes as prodrugs. Multi-action Pt^{IV} prodrugs are usually prepared by oxidative addition of the square planar Pt^{II} drugs with H₂O₂ and subsequent modification of the axial hydroxido ligands. Pt^{IV} complexes are particularity suitable as prodrugs because they are stable outside the cancer cell and become activated by reductive elimination inside the cell. The reduction severs the bonds between the platinum and the axial ligands, thereby regenerating the original Pt^{II} drug and releasing the two axial ligands (as depicted in FIG. 1). The axial ligands of the Pt^{IV} complexes can be utilized as lipophilic moieties that enhance passive cellular uptake, as tumor targeting agents, or as linkers to polymers, nanoparticles, proteins or other delivery agents. In addition, they can also be bioactive agents such as approved drugs, enzyme inhibitors, pathway activators or suppressors, epigenetic modifiers, antimetabolites and others that attack different cellular targets and work in synergy with the Pt^{II} drugs to overcome resistance.

The nature of the linkage between the ligand and the axial hydroxido of Pt^{IV} (carboxylate, carbamate, ether or carbonate) is not usually important when conjugating Pt^{IV} to lipophilic moieties, targeting agents or delivery systems. In contrast, the nature of the linkage is important for multi-action Pt^{IV} prodrugs, since the agents should release the bioactive moieties from the axial positions, in their active forms. This implies that the axial oxygen (Pt-OR) should be an integral part of the bioactive molecule or be part of a linker that can be readily eliminated releasing the active drug. In most multi-action Pt^{IV} prodrugs, the bioactive moieties possess a carboxyl group (FIGS. 1A and 1B). The widespread use of bioactive moieties with carboxyl groups probably stems from the fact that following reduction, the active form of the agent is released.

Drugs such as taxol, doxorubicin, 5-fluoruracil, gemcitabine, topotecan or irinotecan are given in combination with platinum drugs in the clinic. They do not have a carboxylate functionality but have either hydroxy or amine groups (FIG. 1—bottom row). Therefore, a major challenge facing medicinal chemists is the design of novel multi-action Pt^{IV} prodrugs that release drugs that have no carboxylates. Molecules with OH functionalities such as estrogens, combretastatin-A4, 7-hydroxy coumarin or wagonin, NBDHEX, and others were conjugated to the axial positions of Pt^{IV} using different linkers (FIG. 2).

To date, there are no effective protocols for conjugating ligands that do not carry carboxylic acid groups, but rather have one or more hydroxyl or amine functionalities, directly to the axial positions of Pt^{IV}. Therefore, the hydroxy containing active molecules had to be modified to incorporate a carboxyl that can be conjugated to the Pt atom. The hydroxyl groups of the active ligands were linked to the Pt^{IV} either by ester linkages (FIGS. 2A and 2E) or by ether bridges (FIGS. 2B, C, D and F). While ester bonds can be cleaved by enzymes or by hydrolysis to release the active agents, ether bonds are very stable and are unlikely to be severed.

### BACKGROUND ART

[1] Giandomenico, et al., Inorg. Chem. 1995, 34, 1015-1021

[2] Wilson et al., Inorg. Chem. 2011, 50, 3103-3115

[3] Lee et al., J. Am. Chem. Soc. 2012, 134, 12668-12674

### GENERAL DESCRIPTION

In order to enable facile and effective synthesis of novel Pt-based agents, e.g., prodrugs, of active compounds that do not contain carboxylic acid (or carboxylate) functionalities, the inventors developed a novel approach for conjugating such molecules via existing hydroxyl groups or amine groups to the axial positions of Pt^{IV} compounds, in a manner that following reduction of the Pt^{IV} the original actives are released.

In most general terms, for hydroxyl containing actives, this approach involves the formation of a bridge between an axial OH groups of the Pt^{IV} complex and an hydroxyl moiety of the bioactive agent. The innovative synthetic approach is generally depicted in FIG. 3. To demonstrate the facile process of the invention, three Pt^{IV} derivatives of cisplatin with hydroxyl-containing actives have been prepared: gemcitabine (Gem), Paclitaxel (Taxol) and estramustine (EM). Other actives and other cisplatin or generally other Pt^{IV} complexes may be used.

It is a purpose of the invention to provide a method of conjugating or forming a bond between an oxygen atom of an axial hydroxido group (e.g., Pt-OH moiety) in a Pt complex and a hetero atom (O, N or S, respectively) of a hydroxyl-, an amine- or a thiol-containing material (herein the hetero-material), the method comprising reacting the Pt complex with the hetero-material (via the hydroxyl, amine or thiol group), wherein (a) an oxygen atom of the Pt hydroxido group is activated or (b) an oxygen atom of the hydroxyl-containing material, or a nitrogen atom of the amine-containing material, or a sulfur atom of the thiol-containing group is activated. When the hetero-material is activated, it is referred to herein as the “activated material”.

The invention further provides a method of synthesizing a conjugate of at least one Pt complex and at least one hydroxyl-, amine- or thiol-containing material, the method comprising reacting the Pt complex with said at least one hydroxyl-, amine- or thiol-containing material, wherein (a) an oxygen atom of the Pt hydroxido group is activated or (b) an oxygen atom of the hydroxyl-containing material, or a nitrogen atom of the amine-containing material, or a sulfur atom of the thiol-containing group is activated (for association).

Thus, the invention provides a platform approach wherein:
in some embodiments, the method is for conjugating or forming a bond between an oxygen atom of an axial hydroxido group (e.g., Pt-OH moiety) in a Pt complex and an oxygen atom of a hydroxyl-containing material, the method comprising reacting the Pt complex with the hydroxyl-containing material, wherein (a) the oxygen atom of the hydroxido group or (b) the oxygen atom of the hydroxyl-containing material is activated;
in some embodiments, the method is for conjugating or forming a bond between an oxygen atom of an axial hydroxido group (e.g., Pt—OH moiety) in a Pt complex and a nitrogen atom of an amine-containing material, the method comprising reacting the Pt complex with the amine-containing material, wherein (a) the oxygen atom of the hydroxido group or (b) the nitrogen atom of the amine-containing material is activated; or
in some embodiments, the method is for conjugating or forming a bond between an oxygen atom of an axial hydroxido group (e.g., Pt—OH moiety) in a Pt complex and a sulfur atom of a thiol-containing material, the method comprising reacting the Pt complex with the thiol-containing material, wherein (a) the oxygen atom of the hydroxido group or (b) the sulfur atom of the thiol-containing material is activated.

Thus, the method of the invention permits conjugating an active material to a an hydroxido group of a Pt complex, the active material having a hydroxyl oxygen atom, an amine nitrogen atom or a thiol sulfur atom, and the method comprises activating (i) the hydroxide group of the Pt complex or (ii) the hydroxyl oxygen atom, the amine nitrogen atom or the thiol sulfur atom of the active material, to obtain an activated material (the material is the activated for reacting with the non-activated material) and a non-activated material (the material not undergoing activation, as defined); and reacting the activated material and the non-activated material under conditions causing association between the hydroxido group and the oxygen atom, or the nitrogen atom, or the sulfur atom of the active material.

As used herein, the Pt complex is generally of the structure L-Pt-(OH)m, wherein L designates the presence of five ligand groups associated with the Pt atom (wherein 4 of the 5 ligands are in a square planar orientation and one of the 5 ligands is an axially oriented ligand L which may be an hydroxido group), OH is an axially oriented hydroxido group and m is an integer that is either 1 or 2. In cases where m is 1, the axially oriented ligand L is different form OH and where m is 2, the axially oriented ligand L is OH.

The Pt complex may thus be of the general structure (IA) or (IB) below:

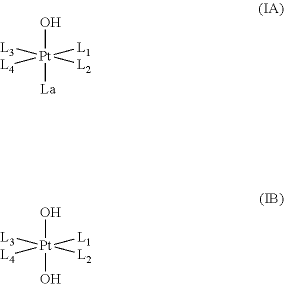

wherein each of L₁, L₂, L₃ and L₄ are planar ligands which may be the same or different and La is an axially oriented ligand which may be different from OH (as in (IA)) or may be OH (as shown in (IB)).

Ligands L₁ through L₄ may be selected amongst such ligands known for Pt complexes. Some of these ligands are depicted in figures of the present application, others are recited, for example, in WO 2015/166498 and US applications derived therefrom, each being fully incorporated herein by reference.

The ligands L may be L is a ligand atom or group of atoms selected from alkyl containing from 1 to 20 carbons, alkenyl containing from 2 to 20 carbons, alkynyl containing from 2 to 20 carbons, cycloalkyl containing from 3 to 10 carbon atoms, cycloalkenyl containing from 3 to 10 carbon atoms, cycloalkynyl containing from 3 to 10 carbon atoms, aryl containing from 6 to 10 carbon atoms, heteroaryl comprising 5 to 15 members wherein 1 to 3 of the atoms in the ring system are a heteroatom selected from nitrogen, oxygen or sulfur, heterocyclyl containing from 3 to 10 members wherein 1 to 3 of the atoms in the ring system are a heteroatom selected from nitrogen, oxygen or sulfur, halide atom, —NR₁R₂, —OR₃, —SR₄, —S(O)R₅, C₂-C₂₀-alkylene-COOH, —OH, —SH, —NH, or any one of the ligands disclosed in WO 2015/166498.

Depending on the nature of the Pt complex to be associated with a hydroxyl-containing material, an amine-containing material or a thiol-containing material, e.g., an active agent or an active material, namely on whether the Pt complex is of the general formula (IA) or (IB), the conjugate or product of a method of the invention has a structure comprising at least one Pt center (being the Pt complex as defined) and at least one or at least two active agent moieties that are bonded thereto via the oxygen atom(s) of the hydroxido group(s). Thus, conjugates or compounds produced by any one method of the invention may be generally depicted as having the structure of formula (IIA) or (IIB):

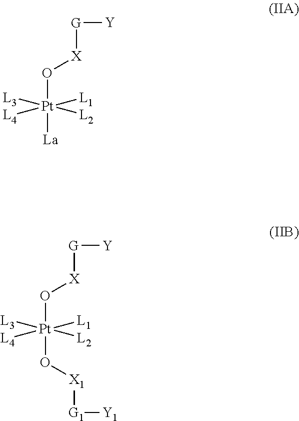

wherein X is a linker atom or a linker group and G-Y and G₁-Y₁ designates a material or an active agent that is covalently associated to X through a native atom G or G1 that is present on the material or the active agent and which is selected from O (in case of hydroxyl-containing materials), N or NH (in case of amine-containing materials) or S (in case of thiol-containing materials). In compounds of the formula (IIB), each of the axial groups may contain the same X atoms or groups of atoms (X=X₁) or different X atoms or groups of atoms (X≠X₁). Additionally, in compounds of the formula (IIB), the active agent designated by Y and Y₁ may be the same or different and atom G and G₁ may be the same or different.

In some embodiments, compounds of the general formula (IIB) are symmetric, namely the moiety -X1-G1-Y1 is the same as moiety -X-G-Y. In some embodiments, compounds of the general formula (IIB) are asymmetric, namely the moiety -X₁-G₁-Y₁ is different from moiety -X-G-Y in at least one of X, X₁, G, G₁, Y or Y₁.

In some embodiments, in compounds of the formula (IIA) or (IIB), independently, G and G1, is oxygen.

In some embodiments, in compounds of the formula (IIA) or (IIB), independently, G and G1, is sulfur.

In some embodiments, in compounds of the formula (IIA) or (IIB), independently, G and G1, is nitrogen (N or NH).

In some embodiments, in an asymmetric compound of the formula (IIB), G may be O and G₁ may be S or N or NH. In some embodiments, G is N or NH and G₁ is O or S.

In some embodiments, in compounds of the formula (IIB), G-Y and G₁-Y₁ are each an hydroxyl-containing material, which may be the same or different.

In some embodiments, in compounds of the formula (IIB), G-Y and G₁-Y₁ are each an amine-containing material, which may be the same or different.

In some embodiments, in compounds of the formula (IIB), G-Y and G₁-Y₁ are each a thiol-containing material, which may be the same or different.

As it is the purpose of the invention to provide multi-action Pt-based anticancer agents which following administration dissociate in the cancer cell into the active Pt complex and an active agent(s), the association of the two via the linker X must be reversible or labile under physiological conditions. According to methods of the invention, either the Pt complex or the active agent are activated in a way which permits the association depicted in the structures of formula (IIA) and (IIB). According to a first methodology, the Pt complex is reacted with an activated hydroxyl-, amine- or thiol-containing material, generally designated HO—Y (a hydroxyl-containing material), H₂N—Y (an amine-containing material), HRN—Y (secondary amine-containing material), or HS—Y (a thiol-containing material) under conditions permitting association of the hydroxido oxygen and the hetero atom on the activated material via a linker atom or a linker group to afford a compound of the formula (IIA) or (IIB). The activated material is of the general form Z-G-Y (or Z-G₁-Y₁), wherein Z is a covalently associated atom or group of atoms and G is the heteroatom (O, N or S) native to the active material Y. In the alternative second methodology, the oxygen atom of the hydroxido group is activated to generally provide the Pt complex in a form L-Pt—O—Z. This activated Pt complex is thereafter reacted with a non-activated hetero-material under conditions permitting association of the hydroxido oxygen and the hetero atom on the hetero-material via a linker atom or a linker group.

The activating group -Z may generally be of the structure -X-A, wherein X is the linker atom or group of atoms that eventually establishes the association between the Pt complex and the hydroxide-containing material and A is a leaving group, as known in synthetic organic chemistry.

Linker X is typically of the structure —C(═O)—, —C(═O)—O—(CH₂)₂S—S(CH₂)₂—O—C(═O)—, —C(═O)—NH—(CH₂)₂S—S(CH₂)₂—O—C(═O)—, —C(═O)—O—(CH₂)₂S—S(CH₂)₂—O—C(═O)—NH—, —C(═O)—O—(CH₂)₂S—S(CH₂)₂—NH—C(═O)— or —C(═O)—NH—(CH₂)₂S—S(CH₂)₂—NH—C(═O)—.

In some embodiments, X is a linker moiety that comprises a Pt atom linking to a hetero material, as defined. Such systems are structured to comprise two or more Pt centers, each center being associated to one or more hetero material.

In cases where the hydroxido group of the Pt complex is activated, it may be of the form L-Pt—O—C(═O)-A or L-Pt—O—C(═O)—O—(CH₂)₂S—S(CH₂)₂—O—C(═O)-A.

In cases where the hetero atom of the hetero material is activated, it may be of the form Y—O—C(═O)-A, Y—NH—C(═O)-A, Y—NR—C(═O)-A, Y—S—C(═O)-A, Y—O—C(═O)—O—(CH₂)₂S—S(CH₂)₂—O—C(═O)-A, Y—NH—C(═O)—O(CH₂)₂S—S(CH₂)₂—O—C(═O)-A, Y—NR—C(═O)—O—(CH₂)₂S—S(CH₂)₂—O—C(═O)-A, Y—S—C(═O)—O—(CH₂)₂S—S(CH₂)₂—O—C(═O)-A, Y—S—C(═O)—NH—(CH₂)₂S—S(CH₂)₂—NH—C(═O)-A, Y—S—C(═O)—NH—(CH₂)₂S—S(CH₂)₂—O—C(═O)-A, Y—S—C(═O)—O—(CH₂)₂S—S(CH₂)₂—NH—C(═O)-A, Y—O—C(═O)—NH—(CH₂)₂S—S(CH₂)₂—O—C(═O)-A, Y—O—C(═O)—O—(CH₂)₂S—S(CH₂)₂—NH—C(═O)-A, Y—NH—C(═O)—NH—(CH₂)₂S—S(CH₂)₂—NH—C(═O)-A or Y—NH—C(═O)—O—(CH₂)₂S—S(CH₂)₂—NH—C(═O)-A.

When the Pt complex is activated or when the hetero material is activated, the conjugate may be of the form L-Pt—O—C(═O)—O—Y, L-Pt—O—C(═O)—NH—Y, L-Pt—O—C(═O)—NR—Y, L-Pt—O—C(═O)—S—Y, L-Pt—O—C(═O)—O—(CH₂)₂S—S(CH₂)₂—O—C(═O)O—Y, L-Pt—O—C(═O)—O—(CH₂)₂S—S(CH₂)₂—O—C(═O)NH—Y, L-Pt—O—C(═O)—O—(CH₂)₂S—S(CH₂)₂—O—C(═O)NR—Y, L-Pt—O—C(═O)—O—(CH₂)₂S—S(CH₂)₂—O—C(═O)S—Y, L-Pt—O—C(═O)—NH—(CH₂)₂S—S(CH₂)₂—O—C(═O)O—Y, L-Pt—O—C(═O)—NH—(CH₂)₂S—S(CH₂)₂—NH—C(═O)O—Y, L-Pt—O—C(═O)—O—(CH₂)₂S—S(CH₂)₂—NH—C(═O)O—Y, L-Pt—O—C(═O)—NH—(CH₂)₂S—S(CH₂)₂—O—C(═O)NH—Y, L-Pt—O—C(═O)—O—(CH₂)₂S—S(CH₂)₂—NH—C(═O)NH—Y, L-Pt—O—C(═O)—NH—(CH₂)₂S—S(CH₂)₂—NH—C(═O)NH—Y, L-Pt—O—C(═O)—NH—(CH₂)₂S—S(CH₂)₂—O—C(═O)NR—Y, L-Pt—O—C(═O)—NH—(CH₂)₂S—S(CH₂)₂—NH—C(═O)NR—Y, L-Pt—O—C(═O)—O—(CH₂)₂S—S(CH₂)₂—NH—C(═O)NR—Y, L-Pt—O—C(═O)—NH—(CH₂)₂S—S(CH₂)₂—O—C(═O)S—Y, L-Pt—O—C(═O)—NH—(CH₂)₂S—S(CH₂)₂—NH—C(═O)S—Y or L-Pt—O—C(═O)—O—(CH₂)₂S—S(CH₂)₂—NH—C(═O)S—Y.

Thus, compounds of formula (IIA) or (IIB) may be selected from compounds having the structure:

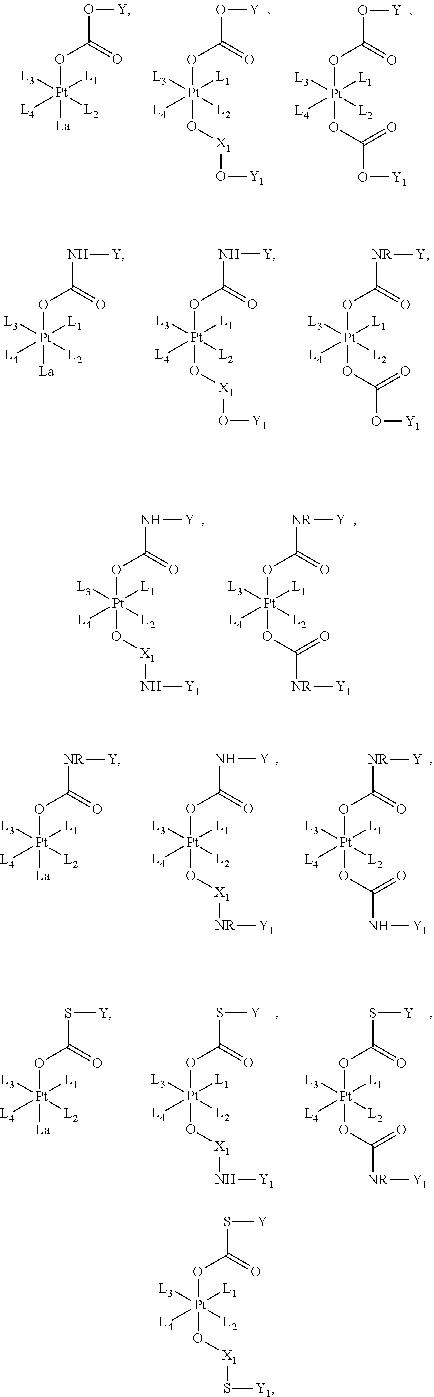

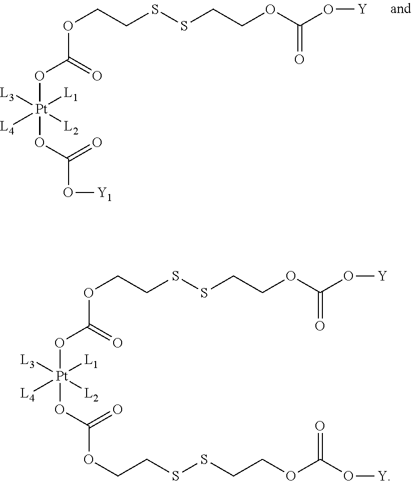

In each of the above structures, ligands L1-L4 are as defined herein; each of X and X1, independently of the other is a linker group or group of atoms as defined herein; each of Y and Y1, independently of the other, is a hetero material having an heteroatom as a point of connectivity; and R is an alkyl or a substituted alkyl on the amine nitrogen atom. In each of the above structure, the heteroatom O, N, S bonded to the Y or Yi moiety (a hetero material) is a heteroatom typically native to the hetero material; namely being a hydroxyl group, an amine or a thiol group of the hetero material.

In some embodiments, the linker group is —C(═O)— forming a carbonate linkage. In some embodiments of a method of the invention, the Pt complex (such as cisplatin, carboplatin and oxaliplatin, or any other Pt complex having a structure as defined herein) having axially oriented hydroxido groups (one or two such groups) is reacted with an active agent with an activating group (thus being ‘activated’) chemically susceptible to substitution by one or both of the axially oriented hydroxido groups.

The “active agent with an activating group” is any hetero material that comprises at least one native hydroxyl, amine or thiol group and which, for the purpose of association to the Pt complex, is substituted to a functionality that enables association to the Pt complex, as detailed herein. For purposes herein, the hetero material is an anticancer drug. In some embodiments, the hetero material is an active material selected from phenols, hormones, hydroxy fatty acids, amine substituted compounds, thiol substituted compounds and others. Non-limiting examples of such agents include combretastatin-A4, 7-hydroxy coumarin, wogonin, 6-(7-nitro-2,1,3-benzoxadiazol-4-ylthio)hexanol (NBDHEX), taxol, estrogen, 5-fluoruracil (5-FU), topotecan, estramustine, gemcitabine, paclitaxel, estramustine, doxorubicin, irinotecan, aniline, dimethylamine, NH₂-derivative of SAHA (Vorinostat—HDACi), derivatives of 3-aminobenzamide and others.

Thus, in a compound of the general formulae below:

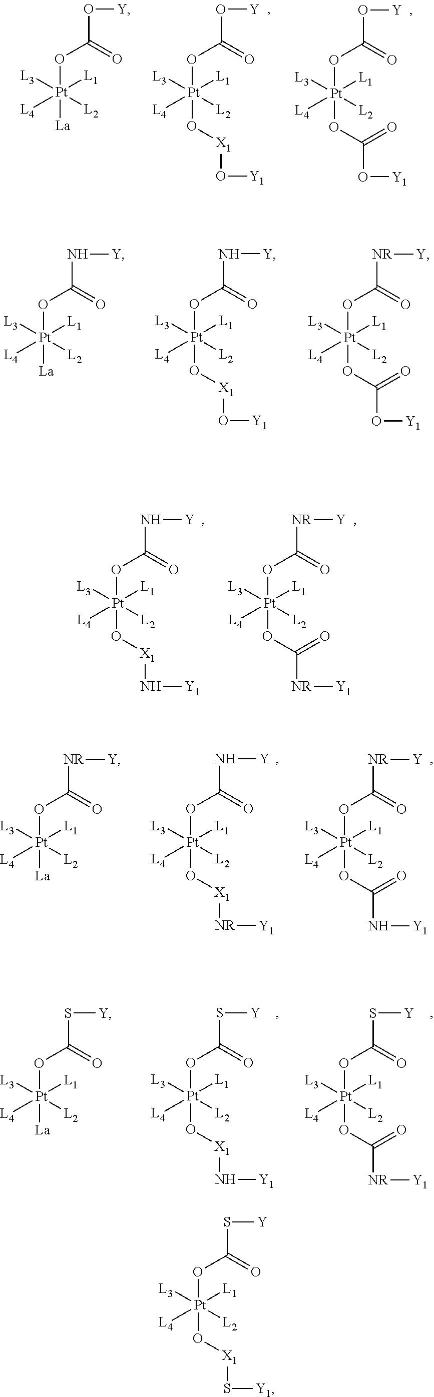

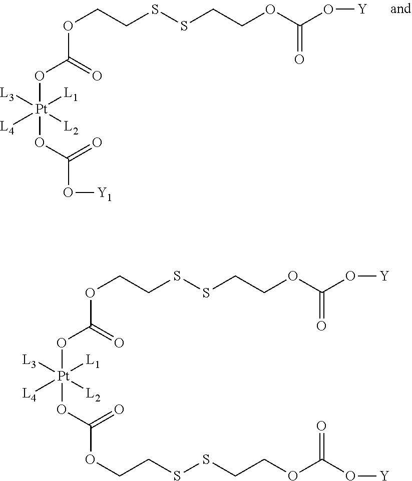

each of Y or Y1 may be, independently, for example, an active agent selected from combretastatin-A4, 7-hydroxy coumarin, wogonin, 6-(7-nitro-2,1,3-benzoxadiazol-4-ylthio)hexanol (NBDHEX), taxol, estrogen, 5-fluoruracil (5-FU), topotecan, estramustine, gemcitabine, paclitaxel, estramustine, doxorubicin and irinotecan, wherein the oxygen atom, nitrogen atom or sulfur atom directly bonded to group Y or Y1 is an atom of the active agent.

Thus, the invention provides a method for the synthesis of a compound of any of the general formulae above, as defined herein, the method comprising reacting a Pt complex of the formula L-Pt—OH or L-Pt—(OH)₂ with an activated active agent, as defined herein, under conditions permitting formation of the compound.

In some embodiments, in a method of the invention, wherein (a) an oxygen atom of the hydroxido group or (b) an oxygen atom of the hydroxyl-containing material, or a nitrogen atom of the amine-containing material, or a sulfur atom of the thiol-containing material is activated, activation is achieved by reacting the Pt complex or the hydroxyl-containing material/amine-containing material/thiol-containing material with a material that enables, upon contact with the non-activated material, covalent association between the two. The activating agent may be any agent known in the art, including N,N′-disuccinimidyl carbonate (DSC) of the structure:

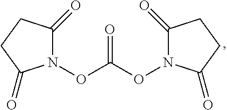 [SMILES: O=C(ON1C(=O)CCC1=O)ON1C(=O)CCC1=O]

phosgene, triphosgene, bisimidazolecarbonyl and others.

Coupling association between the activated material and the non-activated material may proceed under room temperature conditions (a temperature between 13 and 30° C.) or at a temperature between room temperature and 100° C.

An exemplary activation of an amine-containing material is depicted below, wherein each of R, R′ and R″ is a substituting group, and each of R′ and R″ is the amine-containing material, wherein the N atom associated therewith is a native atom thereof.

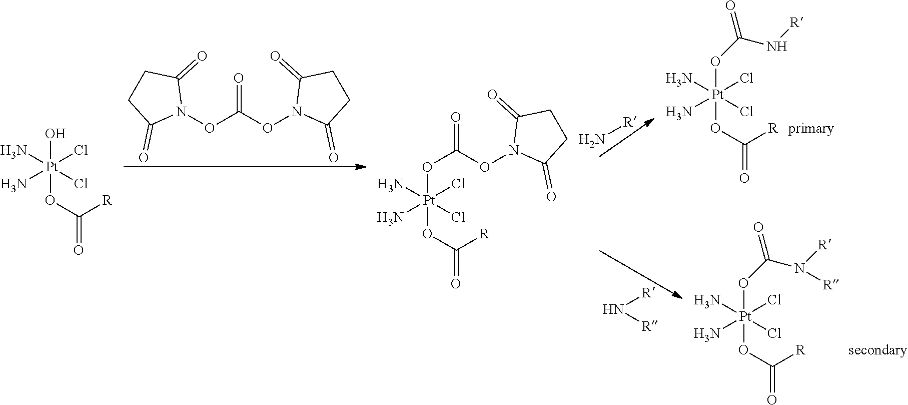

Similar activation of hydroxyl-containing materials is shown below:

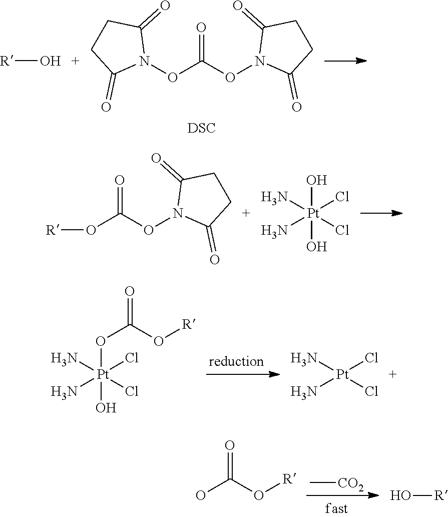

Thus, in some embodiments, the compound obtained may be a product of association of an active drug such as combretastatin-A4, 7-hydroxy coumarin, wogonin, 6-(7-nitro-2,1,3-benzoxadiazol-4-ylthio)hexanol (NBDHEX), taxol, estrogen, 5-fluoruracil (5-FU), topotecan, estramustine, gemcitabine, paclitaxel, estramustine, doxorubicin or irinotecan and a Pt complex of the structure

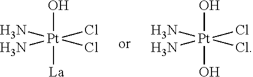

In some embodiments, the product comprises one or more Pt center and one or more active drug. In some embodiments, the product comprises a single Pt center and one or two same or different active drugs.

In some embodiments, the product comprises a single Pt center and a single active drug. In some embodiments, the product comprises a single Pt center and two same or different active drugs.

The invention further provides a compound of any of the general structures:

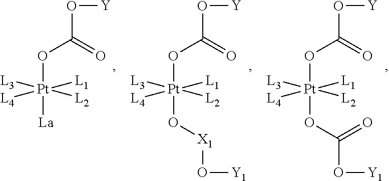

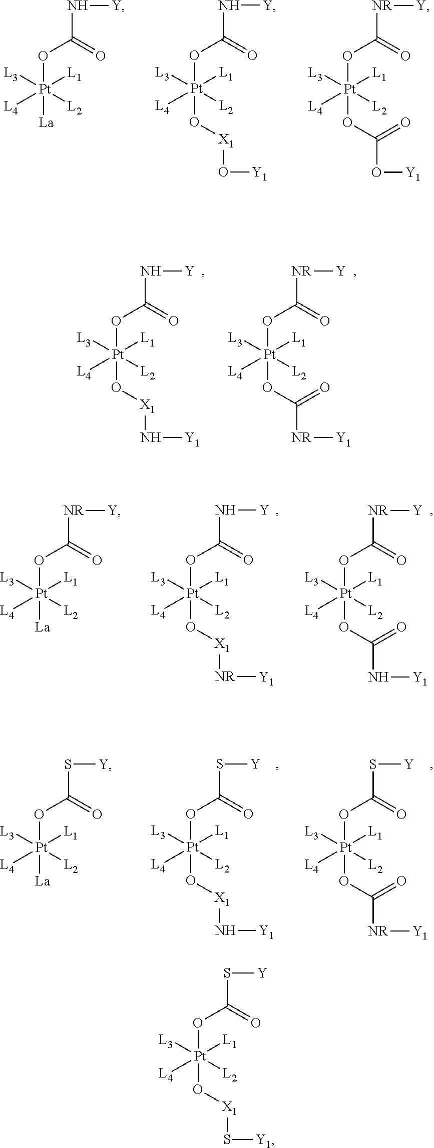

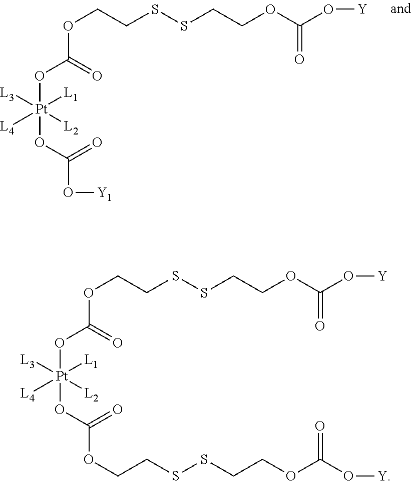

Non-limiting compounds of the invention include:

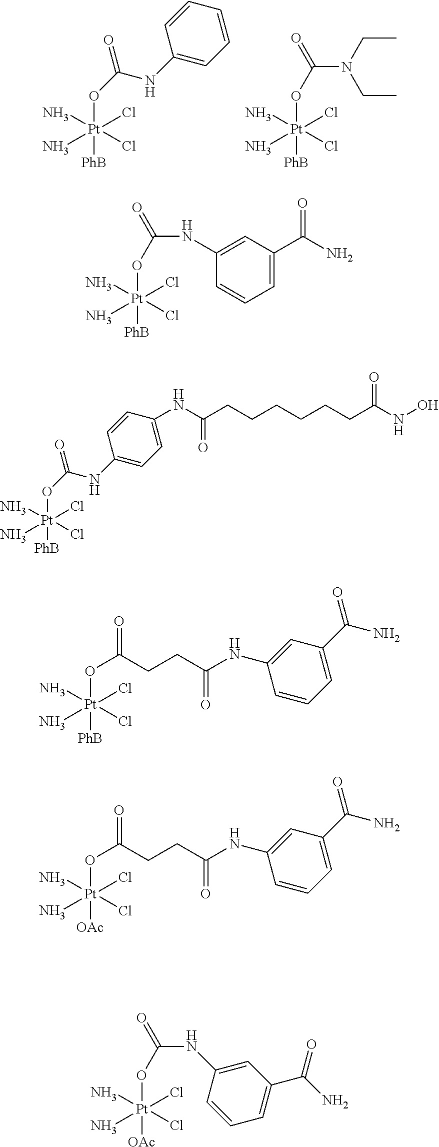

wherein in each of the structures, PhB designates phenylbutyric acid,

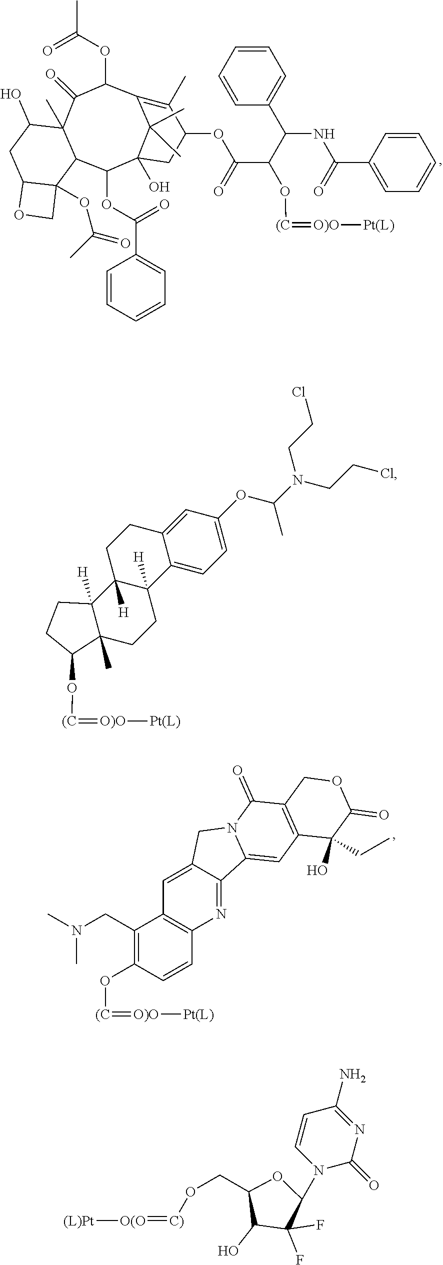

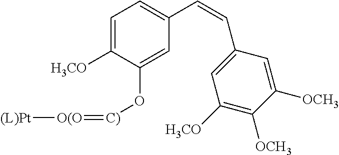

wherein in each of the structures, Pt(L) designates the Pt complex as defined herein.

Also provided are the compounds:

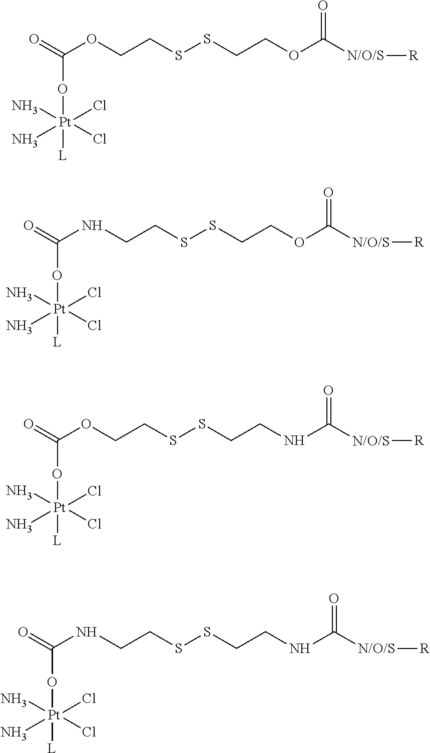

wherein each of the variables is as defined herein.

Also provided are the compounds:

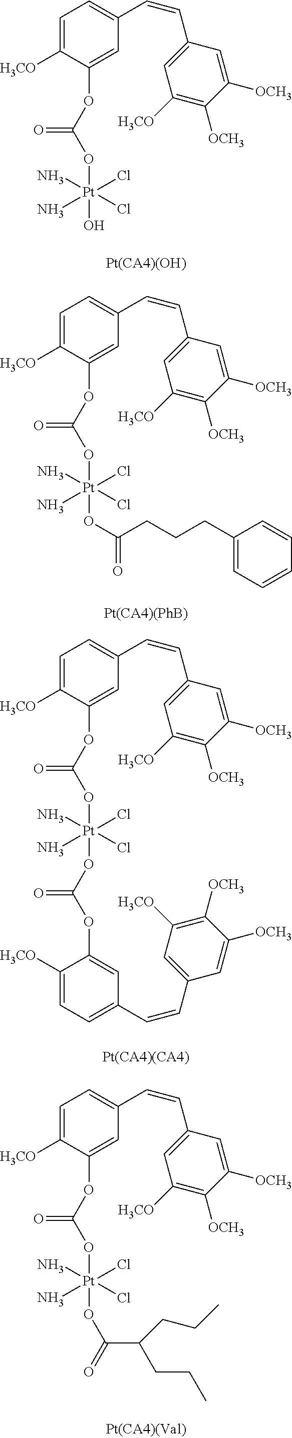

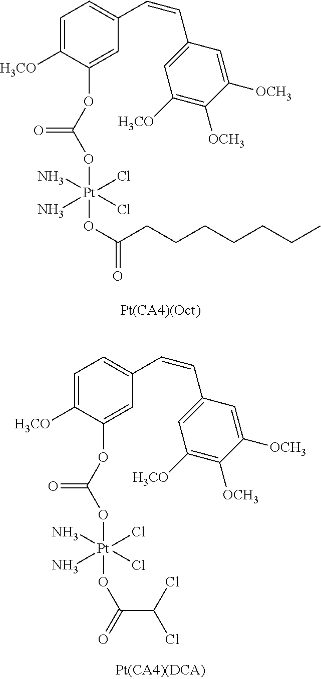

In some embodiments, a compound of the invention is:

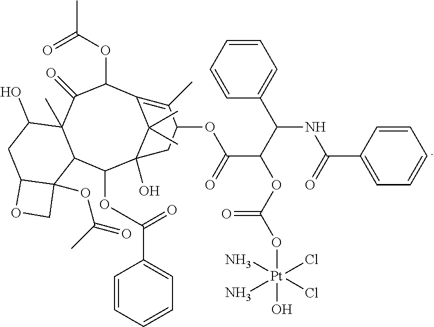

The invention further provides a compound comprising two or more Pt centers, each being of the formula (IA) or (IB), as defined herein, and two or more active drugs, as defined.

In some embodiments, the compound is if the general formula shown below:

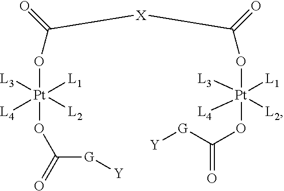

wherein X, G and Y are as defined above,.

In some embodiments, the compound is of the formula:

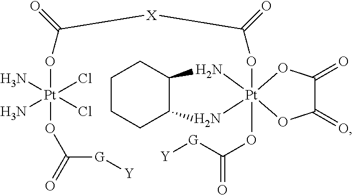

as defined.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1. (top row)—the three FDA approved Pt^{II} drugs: cisplatin, carboplatin and oxaliplatin and dual-action (A) and triple-action (B) Pt^{IV} prodrugs. (middle row)—synthesis and mode of action of Pt^{IV} prodrugs and four anticancer drugs with no carboxylate groups (bottom row).

FIGS. 2A-F. Pt^{IV} prodrugs to which OH containing bioactive ligands were attach by forming an ester link to the OH (A & E) or via an ether linkage (B, C, D & F).

FIG. 3. The synthetic route for conjugating the OH group of a ligand to the axial position of Pt^{IV} via a carbonate linkage (top); the release and activation of the bioactive ligand (middle); and the three Pt^{IV} multi-action prodrugs with gemcitabine, taxol and estramustine.

FIGS. 4A-B. A) the HPLC chromatograms of ctc-[Pt(NH₃)₂(Gem-Carb)(PhB)Cl₂]—top, Gem (middle) and the reaction mixture of ctc-[Pt(NH₃)₂(Gem-Carb)(PhB)Cl₂] with 10 eq of ascorbate after 2 h. B) ¹H NMR spectra of the reduction of ctc-[Pt(NH₃)₂(Tax-Carb)(OH)Cl₂] with 5 eq of ascorbate, monitoring the only amide proton of taxol

FIGS. 5A-B. The reduction of (A) ctc-[Pt(NH₃)₂(EM-Carb)(OAc)Cl₂] compared to (B) the reduction and hydrolysis of the ester linked ctc-[Pt(NH₃)₂(EM-Suc)(OAc)Cl₂].

FIG. 6. Synthetic route for the preparation of complexes ctc-[Pt(NH₃)₂(Gem-Carb)(PhB)Cl₂] & ctc-[Pt(NH₃)₂(Gem-Suc)(PhB)Cl₂].

FIG. 7. Synthetic route for the preparation of ctc-[Pt(NH₃)₂(Tax-Carb)(OH)Cl₂].)

FIG. 8. Synthetic route for the preparation of complexes ctc-[Pt(NH₃)₂(EM-Carb)(OAc)Cl₂] & ctc-[Pt(NH₃)₂(EM-Suc)(OAc)Cl₂]. The numbers indicated in β-estradiol have been used for ¹H & ¹³C NMR numbering of the steroid moiety of synthesized compounds respectively.

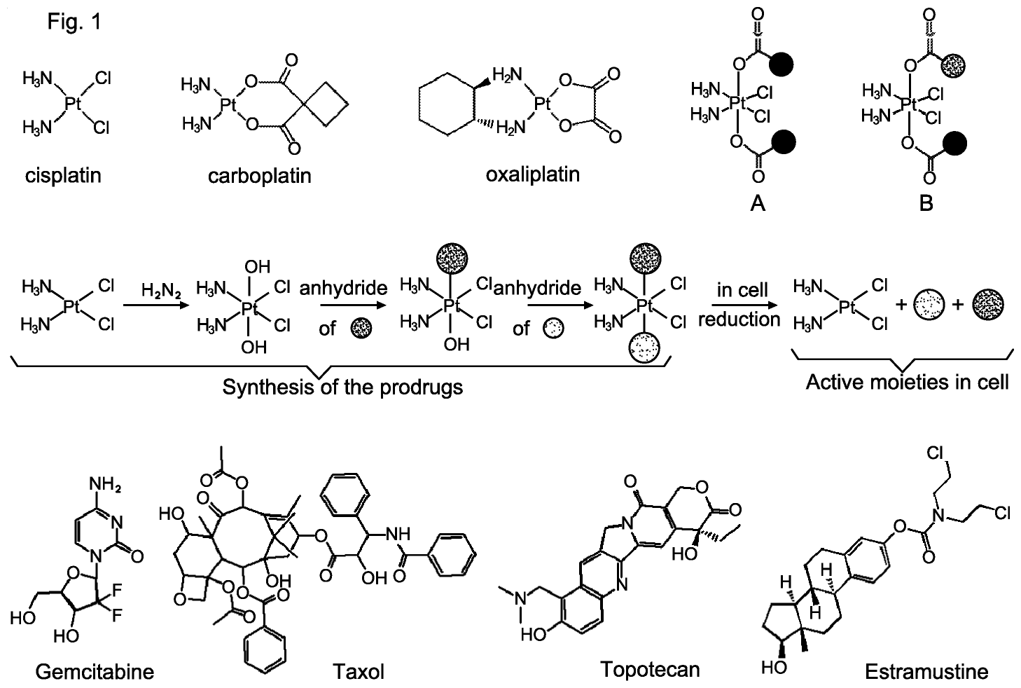

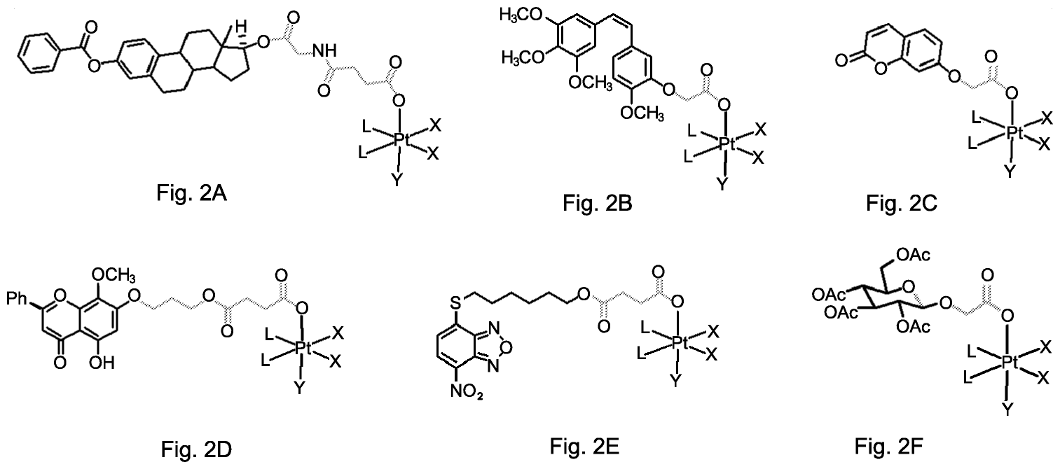

### DETAILED DESCRIPTION OF EMBODIMENTS

### Materials and Methods

All the chemicals and solvents were procured from authentic commercial sources and used without further purification.

The newly synthesized Pt^{IV} compounds were characterized by ¹H NMR, ¹⁹⁵Pt NMR, ESI-MS and elemental analysis. Progress of reactions was monitored by analytical HPLC system (Thermo Scientific UltiMate 3000) with a reverse-phase C18 column (Phenomenex Kinetex, Length 250 mm, Internal dia 4.60 mm, Particle size 5 μm, Pore size 100 Å). The purity and retention time (RT) of synthesized compound reported here were measured with the same analytical HPLC system either water/acetonitrile gradient or TFA (0.1% in water)/acetonitrile gradient at the flow rate of 1 mL/min Reaction mixtures were purified on a preparative HPLC system (Thermo Scientific UltimaMate 3000 station) equipped with a reverse-phase C18 column (Phenomenex Luna 250×21.2 mm, 10 μm, 100 Å) with the similar type of mobile phase was used with the flow rate of 15 mL/min. UV detection was set at 220 nm in both the HPLC systems. The fractions were combined and lyophilized to get the pure compounds.

All NMR data were collected on a Bruker AVANCE III™ HD 500 MHz spectrometer. The data were processed using either MestreNova or Bruker TopSpin 3.6.0 software. ¹H & ¹³C NMR chemical shifts were referenced with the individual solvent residual peaks of respective NMR solvents used. ¹⁹⁵Pt NMR chemical shifts were reported with respect to chemical shift of standard K₂PtCl₄ in water at −1624 ppm. Electrospray ionization mass spectra (ESI-MS) were done using a Thermo Scientific triple quadrature mass spectrometer (Quantum Access) by +ve/−ve mode electrospray ionization. Elemental analyses reported were performed using a Thermo ScientificFLASH 2000 element analyzer.

### Syntheses

### N-Succinimidyl di-Boc Gemcitabine Carbonate (Di-Boc Gem-Carb-NHS)

Initially, the exocyclic amine and the 3′-OH of gemcitabine were protected with di-tert-butyl dicarbonate (Boc anhydride) to yield 4-N-3′-O-bis(tert-butoxycarbonyl)gemcitabine (di-Boc gemcitabine) according to the reported literature protocol.

di-Boc gemcitabine (463 mg, 1.0 mmol) was taken in a mixture of acetonitrile and DCM (1:1 v/v, 10 mL). To this, N,N′-disuccinimidyl carbonate, DSC (332 mg, 1.3 mmol) and 4-(dimethylamino)pyridine, DMAP (122 mg, 1 mmol) were added and stirring was continued at room temperature. After 20 h, the progress of the reaction is monitored by HPLC (a new peak appeared at a RT of 23.1 mins with a programme using 0-90% linear gradient of 0.1% TFA in water to acetonitrile as mobile phase over 30 min). After completion, the solvents were evaporated and the reaction mixture was extracted with excess DCM and water to afford the crude N-succinimidyl di-Boc gemcitabine carbonate (560 mg, 92%). This was used for the next step without any further purification.

ctc-[Pt(NH₃)₂(Gem-Carb)(PhB)Cl₂]: The crude N-succinimidyl di-Boc gemcitabine carbonate (708 mg, 1.2 mmol) was treated with oxoplatin (334 mg, 1 mmol) in DMSO at room temperature. ¹⁹⁵Pt NMR indicated that the reaction was completed after overnight stirring. The DMSO solution was suspended in excess diethyl ether yielding a precipitate which was collected by centrifugation. The precipitate was dissolved in methanol and then was re-precipitated with excess diethyl ether affording ctc-[Pt(NH₃)₂(Di-Boc Gem-Carb)(OH)Cl₂]. Yield: (600 mg, 73%).

To ctc-[Pt(NH₃)₂(Di-Boc Gem-Carb)(OH)Cl₂] (190 mg, 0.23 mmol), phenyl butyric anhydride² (429 mg, 1.38 mmol) was added at once in DMF and the resulting solution was stirred overnight at room temperature. After completion (monitored by ¹⁹⁵Pt NMR), the DMF was evaporated to give a sticky oil which was dissolved in methanol and precipitated with excess diethyl ether. Solid precipitate was recovered by centrifugation and subsequently dried.

The solid was re-dissolved in a mixture of DCM & TFA (1:1, 2 mL) and stirred at room temperature for 45 min (The progress of deprotection was monitored using HPLC). A steady stream of air is applied overnight to remove the solvents and to afford a sticky yellow solid. Finally, this crude mixture was purified over preparative HPLC with 0.1% TFA in water/acetonitrile gradient and lyophilized to obtain the TFA salt of ctc-[Pt(NH₃)₂(Gem-Carb)(PhB)Cl₂] (52 mg, 30% overall yield). RT 15.9 min, ran with 0-90% linear gradient of water to acetonitrile as mobile phase over 30 min, purity >98%. ¹H NMR (500 MHz, MeOD) δ: 8.12 (d, J=7.9 Hz, 1H), 7.29-7.12 (m, 5H), 6.37 (d, J=8.0 Hz, 1H), 6.22-6.14 (m, 1H), 4.53-4.50 (m, 1H), 4.37-4.31 (m, 2H), 4.11 (dt, J=8.8 & 2.6 Hz, 1H), 2.67 (t, J=7.6 Hz, 2H), 2.42 (t, J=7.4 Hz, 2H), 1.91-1.85 (m, 2H); ¹³C NMR (125 MHz, MeOD) δ 183.6, 162.3, 162.0, 161.5, 160.9, 148.8, 145.4, 143.3, 129.7, 129.3, 126.8, 123.5, 96.4, 85.7, 80.6, 70.4, 64.8, 36.2, 35.9, 28.9; ¹⁹⁵Pt NMR (107.5 MHz, MeOD) δ:1113.5; ESI-MS (m/z) calculated for [C₂₀H₂₇Cl₂F₂N₅O₈Pt+H]⁺: 769.09, found 769.76; Elemental analysis (%) calculated for C₂₀H₂₇Cl₂F₂N₅O₈Pt (*1.0H₂O*1.7 TFA): C 28.64, H 3.15, N 7.14, found C 28.71, H 3.17, N 7.12.

Di-Boc gemcitabine succinate (Di-Boc Gem-Sue): Di-Boc gemcitabine (463 mg, 1 mmol), succinic anhydride (500 mg, 5 mmol) and DMAP (24 mg, 0.2 mmol) were taken in DMF (6-7 mL) and were stirred at room temperature for overnight. The progress of the reaction was monitored by HPLC using a linear gradient of 0-90% water to acetonitrile. The starting material elutes at 21.04 min. and the desired product at 21.5 min. After completion (i.e. approximate conversion of over 90%), the solvent is evaporated under reduced pressure after which the crude reaction mixture is re-dissolved in methanol and diethyl ether was used to precipitate impurities from the reaction mixture. The filtrate was collected and combined filtrate fractions were evaporated to dryness. The sticky oily material obtained was used directly for the next step without any further purification.

N-Succinimidyl di-Boc gemcitabine succinate (Di-Boc Gem-Suc-NHS): The crude di-Boc gemcitabine succinate was treated with N,N′-dicyclohecylcarbodiimide, DCC (412 mg, 2.0 mmol), N-hydroxy succinimide, NHS (230 mg, 2.0 mmol) in DMF (6-7 mL) and the reaction mixture is stirred at 40° C. for 20 hours. The progress of reaction is monitored by HPLC (A new peak will appear at a retention time of 23.0 min ran with 0-90% linear gradient of 0.1% TFA in water to acetonitrile as mobile phase over 30 min). After completion, the suspension formed in the flask was filtered, and the filtrate was evaporated to dryness. The crude reaction mixture is purified over preparative HPLC to obtain the N-succinimidyl di-Boc gemcitabine succinate and was well characterized by ¹H NMR, ¹³C NMR and ESI-MS analysis. The overall yield for the two steps (i.e. for the synthesis of di-Boc gemcitabine succinate and the synthesis of N-succinimidyl di-Boc gemcitabine succinate) is around 45% (300 mg) along with the recovery of gemcitabine succinate in 28% (160 mg). ¹H NMR (500 MHz, CDCl₃) δ: 7.67 (dd, J=7.7 & 1.6 Hz, 1H), 7.24 (d, J=7.7 Hz, 1H), 6.37 (dd, J=12.2 & 5.4 Hz, 1H), 5.15-4.95 (m, 1H), 4.58 (dd, J=12.3 & 2.9 Hz, 1H), 4.48-4.29 (m, 2H), 3.09-2.70 (m, 8H), 1.48 (s, 18H); ¹³C NMR (125 MHz, CDCl₃) δ: 171.6, 170.5, 169.0, 167.7, 163.1, 154.2, 151.4, 144.5, 122.3, 120.1, 118.1, 95.8, 84.7, 83.0, 77.7, 73.1, 72.7, 62.3, 48.4, 28.4, 27.9, 27.6, 27.5, 26.1, 25.5; ESI-MS (m/z) calculated for [C₂₇H₃₄F₂N₄O₁₃+H]⁺: 661.22, found 660.99.

ctc-[Pt(NH₃)₂(Gem-Suc)(PhB)Cl₂]: N-Succinimidyl di-Boc gemcitabine succinate (720 mg, 1.1 mmol) & oxoplatin (334 mg, 1.0 mmol) were taken in DMSO and stirred at room temperature. The progress of the reaction is monitored by the ¹⁹⁵Pt NMR where we observed a peak around 1050 ppm corresponding to mono-ester of oxoplatin. After completion, the DMSO was extracted with excess diethyl ether to get an off-white precipitate, which is recovered by centrifugation. Re-dissolving the precipitate in methanol followed by precipitation with excess diethyl ether afforded ctc-[Pt(NH₃)₂(Di-Boc Gem-Suc)(OH)Cl₂].

To ctc-[Pt(NH₃)₂(Di-Boc Gem-Suc)(OH)Cl₂] (150 mg, 0.17 mmol), phenyl butyric anhydride (263 mg, 0.85 mmol) was added at once in DMF and the resulting solution was stirred overnight at room temperature. After completion (by ¹⁹⁵Pt NMR), the DMF was evaporated. The sticky oil obtained was dissolved in methanol and precipitated with excess diethyl ether. The solid precipitate was recovered by centrifugation and subsequently dried.

The solid was re-dissolved in a mixture of DCM and TFA (1:1) and stirred at room temperature for 45 min (The progress of deprotection was monitored using HPLC). A steady stream of air is applied overnight to remove the solvents and to afford a sticky yellow solid. Finally, this crude mixture was purified over preparative HPLC with 0.1% TFA in water/acetonitrile gradient and lyophilized to obtain the TFA salt of ctc-[Pt(NH₃)₂(Gem-Suc)(PhB)Cl₂] (40 mg, 25% overall yield). RT 15.8 min, ran with 0-90% linear gradient of 0.1% TFA in water to acetonitrile as mobile phase over 30 min, purity >97%. ¹H NMR (500 MHz, MeOD) δ: 7.93 (d, J=7.9 Hz, 1H), 7.27-7.12 (m, 5H), 6.27-6.20 (m, 2H), 4.53-4.41 (m, 2H), 4.33-4.25 (m, 1H), 4.24-4.17 (m, 1H), 2.69-2.62 (m, 6H), 2.38 (t, J=7.4 Hz, 2H), 1.91-1.84 (m, 2H); ¹³C NMR (125 MHz, MeOD) δ: 183.9, 181.9, 174.5, 162.4, 150.1, 145.1, 143.4, 129.7, 129.5, 129.3, 126.8, 123.7, 101.4, 96.3, 80.7, 71.4, 63.5, 36.3, 36.2, 31.8, 31.1, 29.0, 20.7; ¹⁹⁵Pt NMR (107.5 MHz, MeOD) δ: 1092.6; ESI-MS (m/z) calculated for [C₂₃H₃₁Cl₂F₂N₅O₉Pt+H]⁺: 825.12, found 825.75; Elemental analysis (%) calculated for C₂₃H₃₁Cl₂F₂N₅O₉Pt (*1.7 H₂O*1.7 TFA): C 30.20, H 3.47, N 6.67, found C 30.15, H 3.48, N 6.66.

N-Succinimidyl paclitaxel carbonate (Tax-Carb-NHS): Paclitaxel (300 mg, 0.35 mmol) was stirred with DSC (900 mg, 3.51 mmol) and DMAP (86 mg, 0.7 mmol) in 40 mL 1:1 mixture of DCM & CH₃CN for 3 hr at room temperature. Later, solvents were evaporated, residue was dissolved in DCM (40 mL) and washed with water (3×30 mL). The organic layer was than dried using anhydrous sodium sulfate and DCM was evaporated. Afterwards, activated paclitaxel was purified using HPLC. RT 13.9 min (0-100% linear gradient of acetonitrile in water over 15 min then 3 min constant at 100% acetonitrile, purity >80%). Yield 200 mg (57%). ¹H NMR (500 MHz, DMSO-d₆) δ: 9.41 (d, 1H, J=8.2 Hz), 8.00 (m, 2H), 7.88 (m, 2H), 7.76 (m, 1H), 7.68 (m, 2H), 7.59 (m, 1H), 7.55 (m, 2H), 7.49 (m, 4H), 7.25-7.22 (m, 1H), 6.29 (s, 1H), 5.87 (m, 1H), 5.64-5.57 (m, 2H), 5.42 (m, 1H), 4.93-4.89 (m, 2H), 4.65 (s, 1H), 4.13-4.06 (m, 1H), 4.04-3.98 (m, 2H), 3.58 (d, 1H, J=7.2 Hz), 2.82 (s, 4H), 2.33-2.27 (m, 1H), 2.25 (s, 3H), 2.11 (s, 3H), 1.84 -1.78 (m, 1H), 1.71 (s, 3H), 1.67 -1.61 (m, 1H), 1.52-1.48 (m, 4H), 1.03 -1.01 (m, 6H); ¹³C NMR (125 MHz, DMSO-d₆) δ: 202.3, 169.7, 169.5, 168.8, 167.8, 166.4, 165.3, 150.9, 138.6, 136.2, 133.9, 133.8, 133.5, 131.7, 129.9, 129.6, 128.9, 128.7, 128.5, 128.3, 127.8, 127.4, 83.6, 80.2, 79.3, 76.6, 75.3, 74.6, 74.5, 71.7, 70.4, 57.4, 53.8, 46.1, 42.9, 36.5, 34.2, 26.3, 25.4, 22.6, 21.3, 20.7, 13.9, 9.8; ESI-MS (m/z) calculated for [C₅₂H₅₄N₂O₁₈+H]⁺: 995.35, found 995.07.

ctc-[Pt(NH₃)₂(Tax-Carb)(OH)Cl₂]: Oxoplatin (26.7 mg, 0.08 mmol) was stirred overnight at room temperature with Tax-Carb-NHS (100 mg, 0.1 mmol) in 3 mL of DMSO. Reaction was monitored through ¹⁹⁵Pt NMR where we observed a peak around 1050 ppm corresponding to mono-substituted of oxoplatin. After completion, the DMSO was extracted with excess diethyl ether to get an off-white precipitate, which is recovered by centrifugation. Re-dissolving the precipitate in methanol followed by precipitation with excess diethyl ether afforded crude ctc-[Pt(NH₃)₂(Tax-Carb)(OH)Cl₂] which was purified by HPLC (RT 12 min, 0-100% linear gradient of acetonitrile in water over 15 min then 3 min constant at 100% acetonitrile, purity >99%). Yield 50 mg (50%). ¹H NMR (500 MHz, DMSO-d6) δ: 9.29 (d, 1H, J=8.5 Hz), 7.99 (m, 2H), 7.89 (m, 2H), 7.75 (m, 1H), 7.69 (m, 2H), 7.56 (m, 1H), 7.49 (m, 2H), 7.44-7.39 (m, 4H), 7.14-7.11 (m, 1H), 6.32 (s, 1H), 6.18-5.91 (br, 6H), 5.83 (m, 1H), 5.45-5.40 (m, 2H), 5.13 (m, 1H), 4.91 (m, 1H), 4.87 (m, 1H), 4.53 (s, 1H), 4.12-4.08 (m, 1H), 4.03-3.98 (m, 2H), 3.59 (d, 1H, J=7.2 Hz), 2.54 (s, 1H), 2.36-2.29 (m, 1H), 2.26 (s, 3H), 2.11 (s, 3H), 1.82 (s, 3H), 1.79-1.72 (m, 1H), 1.65 (m, 1H), 1.49 (s, 3H), 1.39-1.35 (m, 1H), 1.02-1.00 (m, 6H); ¹³C NMR (125 MHz, DMSO-d6) δ: 202.5, 170.4, 169.7, 168.8, 166.0, 165.3, 159.3, 140.3, 137.9, 134.2, 133.5, 132.9, 131.4, 129.9, 129.6, 128.7, 128.6, 128.3, 127.9, 127.6, 127.5, 83.6, 80.2, 76.7, 76.2, 75.3, 74.7, 74.5, 70.5, 70.1, 57.3, 54.3, 46.0, 42.9, 36.5, 29.0, 26.3, 22.7, 21.4, 20.7, 14.2, 9.7; ¹⁹⁵Pt NMR (107.5 MHz, DMSO-d6) δ: 1051.1; ESI-MS (m/z) calculated for [C₄₈H₅₇Cl₂N₃O₁₇Pt—H]⁻: 1211.26, found 1211.91; Elemental analysis (%) calculated for C₄₈H₅₇Cl₂N₃O₁₇Pt (*4.0H₂O): C 44.83, H 5.09, N 3.27, found C 44.49, H 4.87, N 3.12.

Bis(2-chloroethyl)carbamic chloride: Triphosgene (285 mg, 0.96 mmol) & bis(2-chloroethyl)amine hydrochloride (515 mg, 2.88 mmol) are combined in presence of a base (pyridine/Et₃N, 5.76 mmol) in DCM following literature procedures to form bis(2-chloroethyl)carbamic chloride and used for the next reaction without further purification. For the characterization purpose of this reactive intermediate, concentrated reaction mixture was directly charged in silica gel column and eluted with 1:2 petroleum ether/ethyl acetate to obtain the product as yellow oil (R_{f}=0.44). Yield: 55 mg (28.1%). ¹H NMR (500 MHz, CDCl₃) δ: 3.93 (t, 2H, J=6.5 Hz, 1×CH₂N), 3.81 (m, 2H, 1×CHCl & 1×CHN), 3.76 (m, 4H, 1 x CHCl, 1×CHN & 1×CH₂Cl); ¹³C NMR (125 MHz, CDCl₃) δ: 149.6 (COCl), 54.2 (CH₂N), 53.2 (CH₂N), 41.5 (CH₂Cl), 40.9 (CH₂Cl).

Estramustine (EM): To a suspension of β-estradiol (273 mg, 1 mmol) in DCM (15 mL), Bu₄NI (442 mg, 1.19 mmol) and NaOH (400 mg, 10 mmol in 15 mL water) were added and stirred vigorously for about 1 h at room temperature to obtain a transparent biphasic solution mixture. Then freshly prepared bis(2-chloroethyl)carbamic chloride (2.32 mmol in 3 mL DCM) was added and stirred for another 1 h. The colour of the reaction mixture changes to straw yellow. After that, the mixture was diluted with DCM and organic layer was separated. The aqueous layer was extracted with DCM (3×30 mL). Combined organic layers were dried over anhydrous Na2SO4 and evaporated to dryness to yield light yellow oil. It was purified by silica gel column chromatography. Elution with DCM removes pink coloured frontrunners then with 2% MeOH in DCM mixture to give EM as white fluffy solid (R_{f}=0.41; RT 28.2 min, ran with 0-90% linear gradient of water to acetonitrile as mobile phase over 30 min then 5 min constant at 90% acetonitrile). Yield: 366 mg (83%). ¹H NMR (500 MHz, CDCl₃) δ: 7.29 (d, 1H, J=8.5 Hz, H1), 6.89 (dd, 1H, J₁=8.5 Hz, J₂=2.4 Hz, H₂), 6.82 (d, 1H, J=2.3 Hz, H4), 3.85-3.70 (m, 9H, H17 & 2×CH₂CH₂Cl), 3.47 (s, 1H, OH), 2.91-2.82 (m, 2H, H6), 2.34-2.29 (m, 1H, H11), 2.24-2.19 (m, 1H, H9), 2.15-2.08 (m, 1H, H16), 1.97-1.93 (m, 1H, H12), 1.90-1.85 (m, 1H, H7), 1.73-1.66 (m, 1H, H15), 1.55-1.16 (m, 7H, H16, H11, H8, H15, H7, H12 & H14), 0.77 (s, 3H, H18); ¹³C NMR (125 MHz, CDCl₃) δ: 154.7 (CO_{Carbamate}), 148.7 (C3), 138.4 (CS), 138.1 (C10), 126.6 (C1), 121.7 (C4), 118.8 (C2), 82.0 (C17), 51.6 (CH₂N), 51.3 (CH₂N), 50.2 (C14), 44.3 (C9), 43.4 (C13), 42.2 (CH₂Cl), 42.1 (CH₂Cl), 38.7 (C8), 36.8 (C12), 30.7 (C16), 29.7 (C6), 27.2 (C7), 26.4 (C11), 23.3 (C15), 11.2 (C18); ESI-MS (m/z) calculated for [C₂₃H₃₁Cl₂NO₃+H]⁺: 440.18, found 440.08; Elemental analysis (%) calculated for C₂₃H₃₁Cl₂NO₃: C 62.73, H 7.10, N 3.18, found C 62.14, H 6.88, N 3.13.

N-Succinimidyl estramustine carbonate (EM-Carb-NHS): Estramustine (350 mg, 0.79 mmol) was mixed with DSC (202.5 mg, 0.79 mmol) & DMAP (51.5 mg, 0.42 mmol) in DCM (20 mL). The mixture was stirred at room temperature for 1 day to afford a clear solution. DCM was evaporated, residue was loaded in silica gel column and eluted with 10% ethyl acetate in DCM to give desired product (R_{f}=0.75; RT=29.9 min, ran with 0-90% linear gradient of water to acetonitrile as mobile phase over 30 min then 5 min constant at 90% acetonitrile) as white solid. Yield 222 mg (48.1%). Unreacted estramustine was also recovered (150 mg, 43%).¹H NMR (500 MHz, CDCl₃) δ: 7.27 (d, 1H, J=8.5 Hz, H1), 6.89 (dd, 1H, J₁=8.5 Hz, J₂=2.5 Hz, H2), 6.83 (d, 1H, J=2.4 Hz, H4), 4.73-4.69 (m, 1H, H17), 3.85-3.73 (m, 8H, 2×CH₂CH₂Cl), 2.89-2.86 (m, 2H, H6), 2.83 (s, 4H, CH_{2 NHS}), 2.34-2.21 (m, 2H, H11 & H9), 2.03-1.99 (m, 1H, H16), 1.91-1.86 (m, 1H, H12), 1.82-1.75 (m, 2H, H7 & H15), 1.55-1.16 (m, 7H, H16, H11, H8, H15, H7, H12 & H14), 0.89 (s, 3H, H18); ¹³C NMR (125 MHz, CDCl₃) δ: 168.9 (CO_{NHS}), 154.7 (CO_{Carbamate}), 151.6 (CO_{Carbamate}), 148.8 (C3), 138.2 (C5), 137.7 (C10), 126.6 (C1), 121.7 (C4), 118.9 (C2), 90.3 (C17), 51.6 (CH₂N), 51.3 (CH₂N), 49.6 (C14), 44.0 (C9), 43.6 (C13), 42.2 (CH₂Cl), 42.1 (CH₂Cl), 38.3 (C8), 36.8 (C12), 29.6 (C16), 27.3 (C6), 27.1 (C7), 26.1 (C11), 25.7 (CH_{2 NHS}), 23.2 (C15), 12.1 (C18); ESI-MS (m/z) calculated for [C₂₈H₃₄Cl₂N₂O₇+H]⁺: 581.18, found 581.08; Elemental analysis (%) calculated for C₂₈H₃₄Cl₂N₂O₇: C 57.84, H 5.89, N 4.82, found C 58.31, H 6.18, N 4.32.

Estramustine succinate (EM-Suc): To a solution of EM (882 mg, 2 mmol) in CHCl₃ (20 mL), succinic anhydride (402 mg, 4.01 mmol), DMAP (270 mg, 2.21 mmol) and Et₃N (306 μL, 2.2 mmol) were added in a sequence. After stirring for 12 h at room temperature, the concentrated reaction mixture was eluted with 3% MeOH in DCM mixture in a silica gel column to give EM-Suc as white fluffy solid (R_{f}=0.39; RT=28.9 min, ran with 0-90% linear gradient of water to acetonitrile as mobile phase over 30 min then 5 min constant at 90% acetonitrile). Yield: 882 mg (81.5%). ¹H NMR (500 MHz, CDCl₃) δ: 7.27 (d, 1H, J=8.3 Hz, H1), 6.88 (dd, 1H, J₁=8.5 Hz, J₂=2.0 Hz, H2), 6.82 (d, 1H, J=2.0 Hz, H4), 4.73-4.69 (m, 1H, H17), 3.85-3.71 (m, 8H, 2×CH₂CH₂Cl), 2.88-2.85 (m, 2H, H6), 2.70-2.62 (m, 4H, CH_{2 Suc}), 2.31-2.17 (m, 3H, H11, H9 & H16), 1.90-1.85 (m, 2H, H12 & H7), 1.77-1.71 (m, 1H, H15), 1.59-1.24 (m, 7H, H16, H11, H8, H15, H7, H12 & H14), 0.82 (s, 3H, H18); ¹³C NMR (125 MHz, CDCl₃) δ: 177.6 (CO_{Suc-Acid}), 172.3 (CO_{Suc-Ester}), 154.7 (CO_{Carbamate}), 148.7 (C3), 138.3 (C5), 137.9 (C10), 126.6 (C1), 121.6 (C4), 118.7 (C2), 83.3 (C17), 51.6 (CH₂N), 51.3 (CH₂N), 49.9 (C14), 44.1 (C9), 43.1 (C13), 42.1 (CH₂Cl), 42.0 (CH2C1), 38.4 (C8), 36.9 (C12), 29.7 (C16), 29.4 (C6), 29.2 (CH_{2 Suc-Acid}), 27.6 (CH_{2 Suc-Ester}), 27.2 (C7), 26.2 (C11), 23.4 (C15), 12.2 (C18); ESI-MS (m/z) calculated for [C₂₇H₃₅Cl₂NO₆—H]⁻: 538.17, found 538.04; Elemental analysis (%) calculated for C₂₇H₃₅Cl₂NO₆: C 60.00, H 6.53, N 2.59, found C 58.55, H 6.49, N 2.49.

N-Succinimidyl estramustine succinate (EM-Suc-NHS): To a solution of EM-Suc (170 mg, 0.31 mmol) in DCM (10 mL), N-hydroxy succinimide (44 mg, 0.38 mmol) and EDC. HCl (72 mg, 0.375 mmol) were added successively at ice-cold condition. Then the solution stirred for 12 h at room temperature. After evaporation of the reaction mixture resulting oil was charged in a silica gel column and eluted with DCM to eliminate frontrunners. Then elution with 5:1 DCM/ethyl acetate gave EM-Suc-NHS as white fluffy solid (R_{f}=0.75; RT=30.2 min, ran with 0-90% linear gradient of water to acetonitrile as mobile phase over 30 min then 5 min constant at 90% acetonitrile). Yield: 190 mg (94.8%). ¹H NMR (500 MHz, CDCl₃) δ: 7.27 (d, 1H, J=8.3 Hz, H1), 6.88 (dd, 1H, J₁=8.5 Hz, J_{2 =2.0} Hz, H2), 6.82 (d, 1H, J=2.0 Hz, H4), 4.75-4.72 (m, 1H, H17), 3.85-3.72 (m, 8H, 2×CH₂CH₂Cl), 2.98-2.95 (m, 2H, H6), 2.87-2.74 (m, 8H, CH_{2 Suc} & CH_{2 NHS}), 2.34-2.18 (m, 3H, H11, H9 & H16), 1.90-1.85 (m, 2H, H12 & H7), 1.77-1.71 (m, 1H, H15), 1.61-1.24 (m, 7H, H16, H11, H8, H15, H7, H12 & H14), 0.82 (s, 3H, H18); ¹³C NMR (125 MHz, CDCl₃) δ: 171.1 (CO_{Suc-NHS}), 169.1 (CO_{Suc-EM}), 167.9 (CO_{NHS}), 154.7 (CO_{Carbamate}), 148.8 (C3), 138.3 (C5), 137.9 (C10), 126.6 (C1), 121.7 (C4), 118.8 (C2), 83.6 (C17), 51.6 (CH₂N), 51.3 (CH2N), 49.9 (C14), 44.1 (C9), 43.2 (C13), 42.2 (CH₂Cl), 42.1 (CH₂Cl), 38.4 (C8), 37.0 (C12), 29.7 (C16), 29.1 (C6), 27.7 (CH_{2 Suc-NHS}), 27.2 (C7), 26.6 (CH_{2 Suc-EM}), 26.2 (C11), 25.8 (CH_{2 NHS}), 23.4 (C15), 12.3 (C18); ESI-MS (m/z) calculated for [C₃₁H₃₈Cl₂N₂O₈+H]⁺: 637.21, found 637.01; Elemental analysis (%) calculated for C₃₁H₃₈Cl₂N₂O₈: C 58.40, H 6.01, N 4.39, found C 57.48, H 6.02, N 4.41.

ctc-[Pt(NH₃)₂(EM-Carb)(OAc)Cl₂]: To a suspension of oxoplatin (40 mg, 0.12 mmol) in DMSO (15 mL), solution of EM-Carb-NHS (100 mg, 0.17 mmol) in DMSO (5 mL) was added. After 3 days of stirring at (30-40)° C., the mixture was centrifuged to remove the unreacted oxoplatin. The filtrate was extracted with diethyl ether to get rid of DMSO. The yellow residue was further reacted with acetic anhydride (170 μL, 1.8 mmol) in DMF (5 mL) for 12 h at room temperature. The reaction mixture was evaporated at reduced pressure to give yellow oil, dissolved in minimum volume of acetonitrile and precipitated with diethyl ether. Finally it was purified by HPLC to obtain ctc-[Pt(NH₃)₂(EM-Carb)(OAc)Cl₂] (RT 25.2 min, ran with 0-90% linear gradient of water to acetonitrile as mobile phase over 30 min, purity >99%) as off white fluffy solid. Yield: 26.5 mg (25.8%). ¹H NMR (500 MHz, Acetone-d₆) δ: 7.29 (d, 1H, J=8.5 Hz, H1), 6.92 (dd, 1H, J₁=8.5 Hz, J_{2 =2.2} Hz, H2), 6.85 (d, 1H, J=2.2 Hz, H4), 6.59-6.39 (br, 6H, NH₃), 4.48-4.44 (m, 1H, H17), 3.89-3.74 (m, 8H, 2×CH₂CH₂Cl), 2.87-2.79 (m, 2H, H6), 2.36-2.29 (m, 1H, H11), 2.26-2.16 (m, 2H, H9 & H16), 1.95-1.88 (m, 5H, H12, COCH3& H7), 1.75-1.69 (m, 1H, H15), 1.60-1.49 (m, 1H, H16), 1.47-1.26 (m, 6H, H11, H8, H15, H7, H12 & H14), 0.83 (s, 3H, H18); ¹³C NMR (125 MHz, Acetone-d₆) δ: 180.4 (COCH₃), 162.1 (CO_{Carbonate}), 155.1 (CO_{Carbonate}), 150.2 (C3), 138.7 (C5), 138.2 (C10), 126.9 (C1), 122.5 (C4), 119.8 (C2), 86.3 (C17), 51.1 (CH₂N), 50.9 (CH₂N), 50.6 (C14), 44.9 (C9), 43.7 (C13), 42.9 (CH₂Cl), 42.1 (CH₂Cl), 39.3 (C8), 37.9 (C12), 29.7 (C16), 28.7 (C6), 27.8 (C7), 27.0 (C11), 23.8 (C15), 22.7 (COCH₃), 12.5 (C18); ¹⁹⁵Pt NMR (107.5 MHz, Acetone-d₆) δ: 1151.0; ESI-MS (m/z) calculated for [C₂₆H₃₉Cl₄N₃O₇Pt—H]⁻: 841.11, found 841.90; Elemental analysis (%) calculated for C₂₆H₃₉Cl₄N₃O₇Pt: C 37.07, H 4.67, N 4.99, found C 36.08, H 4.74, N 4.74.

ctc-[Pt(NH₃)₂(EM-Suc)(OAc)Cl₂]: Oxoplatin (78 mg, 0.23 mmol) was suspended in DMSO (2 mL) and solution of EM-Suc-NHS (180 mg, 0.28 mmol) in 6 mL DMSO was added to it. After stirring for 1 day at room temperature, the reaction mixture was centrifuged. Residue was discarded and DMSO of filtrate was extracted several times with diethyl ether to obtain yellow oil. This was dissolved in minimum volume of acetone and precipitated with diethyl ether as light yellow solid of cct-[Pt(NH₃)₂Cl₂(EM-Suc)(OH)]. Yield 150 mg (76%). This solid (100 mg, 0.11 mmol) was dissolved in DMF (5 mL), acetic anhydride (208 μL, 2.2 mmol) was added to it and stirred for 12 h at room temperature. The reaction mixture was evaporated at reduced pressure to give yellow oil, dissolved in minimum volume of acetonitrile and precipitated with diethyl ether. Finally it was purified by HPLC to obtain ctc-[Pt(NH₃)₂(EM-Suc)(OAc)Cl₂] (RT 26.3 min, ran with 0-90% linear gradient of water to acetonitrile as mobile phase over 30 min then 15 min constant at 90% acetonitrile, purity >99%) as light yellow powder. Yield: 65 mg (61.9%). ¹H NMR (500 MHz, Acetone-d₆) δ: 7.29 (d, 1H, J=8.5 Hz, H1), 6.92 (dd, 1H, J₁=8.5 Hz, J₂=2.1 Hz, H2), 6.85 (d, 1H, J=2.1 Hz, H4), 6.55-6.24 (br, 6H, NH₃), 4.72-4.68 (m, 1H, H17), 3.89-3.74 (m, 8H, 2×CH₂CH₂Cl), 2.86-2.84 (br, 2H, H6), 2.59-2.52 (m, 4H, CH_{2 Suc}), 2.36-2.31 (m, 1H, H11), 2.27-2.14 (m, 2H, H9 & H16), 1.96-1.87 (m, 5H, H12, COCH₃ & H7), 1.79-1.73 (m, 1H, H15), 1.63-1.56 (m, 1H, H16), 1.51-1.29 (m, 6H, H11, H8, H15, H7, H12 & H14), 0.87 (s, 3H, H18); ¹³C NMR (125 MHz, Acetone-d₆) δ: 181.8 (CO_{Suc-Pt}), 180.5 (COCH₃), 173.5 (CO_{Suc-EM}), 155.1(CO_{Carbamate}), 150.2 (C3), 138.7 (C5), 138.2 (C10), 126.9 (C1), 122.6 (C4), 119.8 (C2), 83.4 (C17), 51.1 (CH₂N), 50.9 (CH₂N), 50.6 (C14), 44.9 (C9), 43.8 (C13), 42.9 (CH₂Cl), 42.1 (CH₂Cl), 39.3 (C8), 37.8 (C12), 31.7 (CH_{2 Suc-Pt}), 31.1 (CH_{2 Suc-EM}), 30.1 (C16), 28.3 (C6), 27.8 (C7), 26.9 (C11), 23.9 (C15), 22.9 (COCH₃), 12.6 (C18); ¹⁹⁵Pt NMR (107.5 MHz, Acetone-d₆) δ: 1135.4; ESI-MS (m/z) calculated for [C₂₉H₄₃Cl₄N₃O₈Pt+H]⁺: 899.15, found 898.90; Elemental analysis (%) calculated for C₂₉H₄₃Cl₄N₃O₈Pt: C 38.76, H 4.82, N 4.68, found C 38.24, H 4.75, N 4.65.

### Reduction Studies

All the reduction studies of reported Pt^{IV} complexes were performed in presence of 10-20 equivalents of ascorbic acid at 37° C. in dark and monitored by analytical HPLC. ctc-[Pt(NH₃)₂(Gem-Carb)(PhB)Cl₂] & ctc-[Pt(NH₃)₂(Gem-Suc)(PhB)Cl₂] were dissolved in 100 mM phosphate buffer (pH 7.0). They were reacted with 10 equivalents of ascorbic acid respectively and HPLC were ran with 0-90% linear gradient of water (0.1% TFA in water for succinate linked Pt^{IV} complex) to acetonitrile as mobile phase over 30 min. ctc-[Pt(NH₃)₂(Tax-Carb)(OH)Cl₂] was reacted with 20 equivalents of ascorbic acid in 3:2 mixture of phosphate buffer (100 mM, pH 7.0) & MeOH and HPLC were ran with 0-100% linear gradient of acetonitrile in water over 15 min then 3 min constant at 100% acetonitrile. The reduction of ctc-[Pt(NH₃)₂(EM-Carb)(OAc)Cl₂] & ctc-[Pt(NH₃)₂(EM-Suc)(OAc)Cl₂] was performed in 3:2 mixture of phosphate buffer (100 mM, pH 7.0) & MeCN in presence of 10 equivalents of ascorbic acid respectively. The HPLC gradient were used as 0-90% linear gradient of water (0.1% TFA in water for succinate linked Pt^{IV} complex) to acetonitrile as mobile phase over 30 min then 5 min constant at 90% acetonitrile.

The half-lives (t_{1/2}) of ctc-[Pt(NH₃)₂(EM-Carb)(OAc)Cl₂], ctc-[Pt(NH₃)₂(EM-Suc)(OAc)Cl₂] & EM-Suc were obtained after linear fitting of In(Aₜ/A₀) vs time (t) by considering a pseudo first order rate equation (Aₜ=A₀e^{-kt}), where A₀ and Aₜ are the integrated areas of HPLC peaks of the respective complexes at t=0 and at time t respectively and k (slope) is the rate constant. Then the t_{1/2} was calculated by utilizing the equation t_{1/2}=0.693/k.

### NMR Reduction of ctc-[Pt(NH₃)₂(Tax-Carb)(OH)Cl₂]

ctc-[Pt(NH₃)₂(Tax-Carb)(OH)Cl₂] (3 mg, 0.0025 mmol) was dissolve in 750 μL of DMSO-d₆ and mixed with ascorbic acid (2.2 mg, 0.0125 mmol). The ¹H NMR spectra were recorded every 6 min at 37° C.

### Biological Studies

### In Vitro Study

Platinum(IV) complexes were dissolved in DMSO to stock solutions of 1 mg mL⁻¹ just before the experiment, and a calculated amount of drug solution was added to the cell growth medium to a final solvent concentration of 0.5%, which had no discernible effect on cell killing Cisplatin and Gemcitabine (Gem) were dissolved just before the experiment in a 0.9% NaCl solution. Cisplatin, Gem and MTT (3-(4,5-dimethylthiazol-2-yl)-2,5-diphenyltetrazolium bromide) were obtained from Sigma Chemical Co., St Louis, USA.

Cell cultures. The human ovarian A2780 and A2780cisR cancer were obtained from the American Type Culture Collection (ATCC, Rockville, Md.). Cell lines were maintained in the logarithmic phase at 37° C. under a 5% carbon dioxide atmosphere using RPMI 1640 medium containing 10% fetal calf serum, antibiotics (50 units per mL penicillin and 50 μg mL⁻¹ streptomycin) and 2 mM L-glutamine. MTT assay. The growth inhibitory effect towards human cell lines was evaluated by means of MTT (tetrazolium salt reduction) assay. Briefly, 3-8×10³ cells per well, dependent upon the growth characteristics of the cell line, were seeded in 96-well microplates in growth medium (100 μL) and then incubated at 37° C. under a 5% carbon dioxide atmosphere. After 24 h, the medium was removed and replaced with a fresh one containing the compound to be studied at an appropriate concentration. Triplicate cultures were established for each treatment. After 72 h, each well was treated with 10 μL of a 5 mg mL⁻¹ MTT (3-(4,5-dimethylthiazol-2-yl)-2,5-diphenyltetrazoliumbromide) solution, and after additional 5 h, 100 μL of a sodium dodecyl sulfate (SDS) solution in 0.01 M HCl were added. Following overnight incubation, the inhibition of cell growth induced by the tested complexes was detected by measuring the absorbance of each well at 570 nm using a Bio-Rad 680 microplate reader (Bio-Rad, Hercules, CA).The mean absorbance for each drug dose was expressed as a percentage of the control untreated well absorbance and plotted vs. drug concentration. IC₅₀ values represent the drug concentrations that reduce the mean absorbance at 570 nm to 50% of those in the untreated control wells.

### In Vivo Study

In vivo anticancer activity toward Lewis lung carcinoma (LLC). All studies involving animal testing were carried out in accordance with ethical guidelines for animal research acknowledging the European Directive 2010/63/UE as to the animal welfare and protection and the related codes of practice. The mice were purchased from Charles River, housed in steel cages under controlled environmental conditions (constant temperature, humidity, and 12 h dark/light cycle), and alimented with commercial standard feed and tap water ad libitum. The LLC cell line was purchased from ECACC, United Kingdom. The LLC cell line was maintained in DMEM (Euroclone) supplemented with 10% heat inactivated fetal bovine serum (Euroclone), 10 mM L-glutamine, 100 U mL-1 penicillin, and 100 μg mL⁻¹ streptomycin in a 5% CO₂ air incubator at 37° C. The LLC was implanted intramuscularly (i.m.) as a 2×10⁶ cell inoculum into the right hind leg of 8 week old male and female C57BL mice (24±3 g body weight). After 7 days from tumor implantation (visible tumors), mice were randomly divided into 4 groups (5 animals per group) and subjected to daily i.p. administration. Control mice received the vehicle (0.5% DMSO (v/v) and 99.5% of a saline solution (v/v)), whereas treated groups received daily doses of ctc-[Pt(NH₃)₂(Gem-Carb)(PhB)Cl₂] (20 mg kg⁻¹ in vehicle solution, orally) gembitabine (60 mg kg⁻¹ in 0.9% saline solution, iv) or cisplatin (3 mg kg⁻¹ in saline solution, iv). At day 15, animals were sacrificed, the legs were amputated at the proximal end of the femur, and the inhibition of tumor growth was determined from the difference in the weights of the tumor-bearing leg and the healthy leg of the animals, expressed as a percentage referenced to the control animals Body weight was measured at day 0 and from day 7 onwards every 2 days and was taken as a parameter for systemic toxicity. All presented values are the means ±SD of no less than three measurements.

### Reduction of the Pt^{IV} Prodrugs

To test whether the reduction of the carbonate linked molecules leads to the release of the drugs, we exposed ctc-[Pt(NH₃)₂(Gem-Carb)(PhB)Cl₂], ctc-[Pt(NH₃)₂(Tax-Carb)(OH)Cl₂] and ctc-[Pt(NH₃)₂(EM-Carb)(OAc)Cl₂] to an excess of ascorbic acid at 37° C. in phosphate buffer (pH=7.0) and monitored the reduction by HPLC. In addition we monitored the reduction of ctc-[Pt(NH₃)₂(Tax-Carb)(OH)Cl₂] by ¹H NMR.

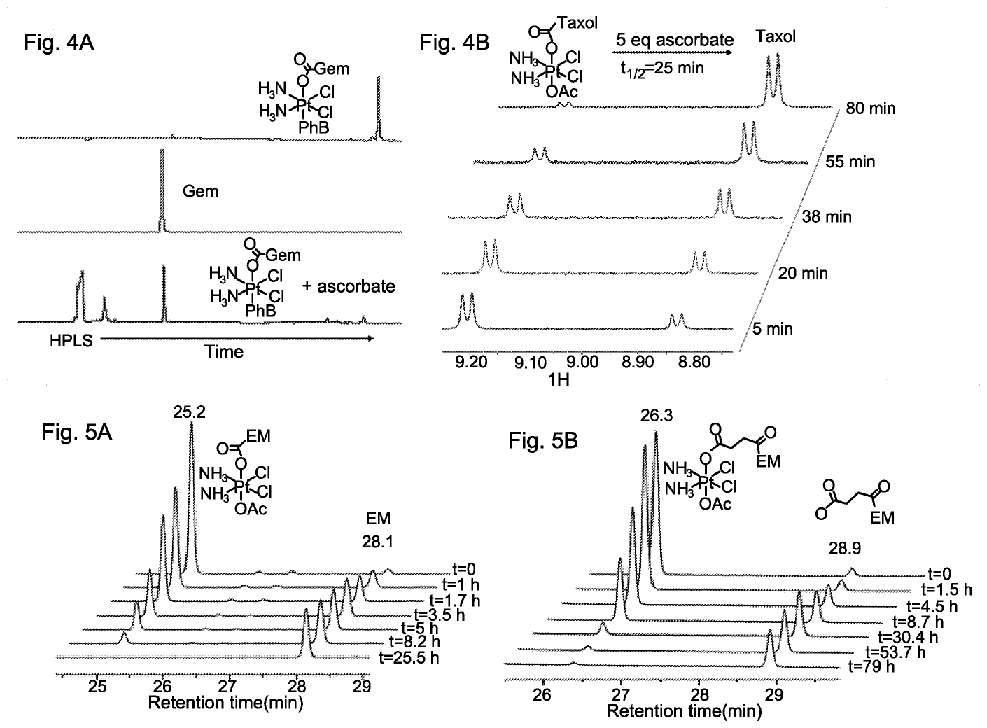

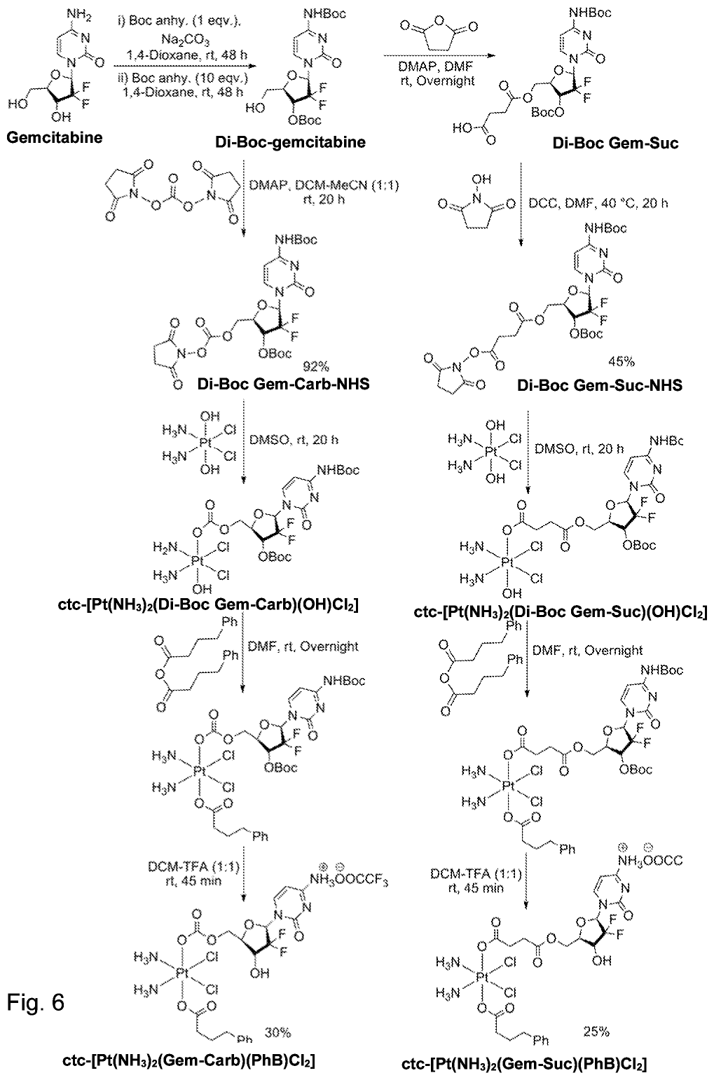

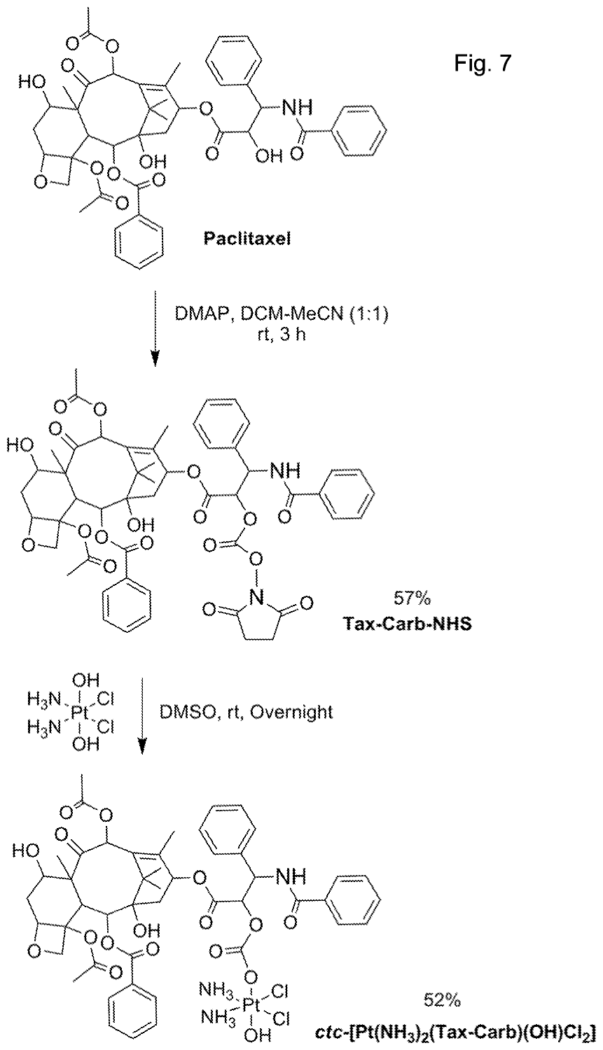

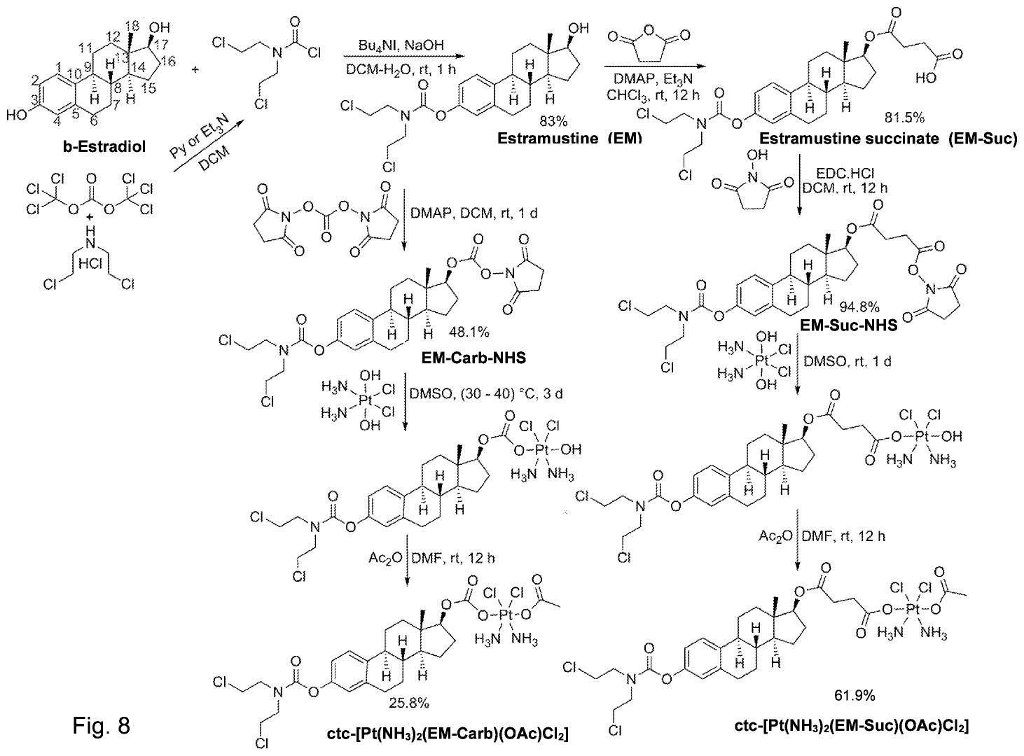

After 2 h, the peak of ctc-[Pt(NH₃)₂(Gem-Carb)(PhB)Cl₂] is gone, and a new peak with the same retention time and the same ESI-MS as Gem appears, indicating complete reduction of ctc-[Pt(NH₃)₂(Gem-Carb)(PhB)Cl₂] and full release of Gem (FIG. 4A). We incubated ctc-[Pt(NH₃)₂(Tax-Carb)(OH)Cl₂] with 5 eq of ascorbate and the reduction was followed by ¹H NMR. The NH proton in ctc-[Pt(NH₃)₂(Tax-Carb)(OH)Cl₂] resonates at 9.2 ppm and in free taxol at ˜8.85 ppm. The reduction of ctc-[Pt(NH₃)₂(Tax-Carb)(OH)Cl₂] with the concurrent growth of Taxol is depicted in FIG. 4B with a half-life of 25 min. No intermediates were observed in the spectra confirming the rapid decarboxylation. Similar results were obtained for the reduction of ctc-[Pt(NH₃)₂(EM-Carb)(OAc)Cl₂] (FIG. 5A).

### Carbonate vs. Succinate Linkers

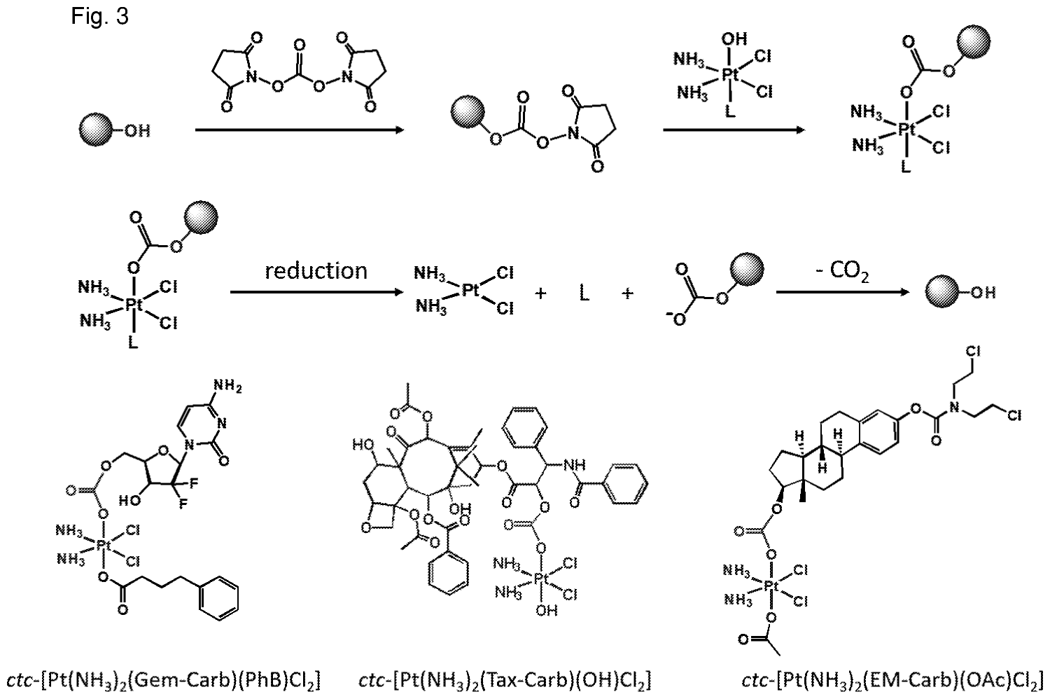

One approach to the preparation of multi-action Pt^{IV} prodrugs with OH containing bioactive molecules is to tether the OH of the drug to the Pt^{IV} via a succinate bridge (FIG. 3). Following reduction, the succinated-drug is released. The ester bond tethering the succinate to the drug has to be severed either by hydrolysis or by esterases to release the active moiety. We prepared ctc-[Pt(NH₃)₂(Gem-Suc)(PhB)Cl₂] and ctc-[Pt(NH₃)₂(EM-Suc)(OAc)Cl₂], the Pt^{IV} succinate-linked analogs of the ctc-[Pt(NH₃)₂(Gem-Carb)(PhB)Cl₂] and ctc-[Pt(NH₃)₂(EM-Carb)(OAc)Cl_{2]} and monitored their reduction, and the release of Gem-Suc or EM-Suc by HPLC (FIG. 5B).

One hour after the reduction of ctc-[Pt(NH₃)₂(EM-Carb)(OAc)Cl₂] began, free EM is observed, and after about 8 h the reduction was nearly complete with concurrent release of EM with RT=28.1 min. (FIG. 5A). In contrast, the reduction of ctc-[Pt(NH₃)₂(EM-Suc)(OAc)Cl₂] yielded only the EM-succinate conjugate with RT=28.9 min, and even after 79 h no free EM was observed (FIG. 5B). The t_{1/2} for the reduction of ctc-[Pt(NH₃)₂(EM-Carb)(OAc)Cl₂] is 2.2 h and for the reduction of ctc-[Pt(NH₃)₂(EM-Suc)(OAc)Cl₂] is 6 h.

Free EM can be generated from ctc-[Pt(NH₃)₂(EM-Suc)(OAc)Cl₂] only by hydrolysis of the ester bond between the EM and the succinate. Since the reduction of ctc-[Pt(NH₃)₂(EM-Suc)(OAc)Cl₂] was nearly complete after 30 h and EM was not observed even after 79 h, we suggest that the rate determining step for the release of EM is the slow hydrolysis of the ester. To confirm this, we prepared the EM-succinate conjugate and monitored the hydrolysis of the ester bond. The t_{1/2} for this hydrolysis was 15 d.

### Anticancer Activity

We chose to combine Gem and taxol with cisplatin because they are potent anticancer drugs, often administered in the clinic with platinum drugs. Preliminary cytotoxicity data against ovarian cancer cells (A2780) and the cisplatin resistant line (A2780cisR) for ctc-[Pt(NH₃)₂(Gem-Carb)(PhB)Cl₂], ctc-[Pt(NH₃)₂(Gem-Suc)(PhB)Cl₂] and ctc-[Pt(NH₃)₂(Tax-Carb)(OH)Cl₂] together with the controls (cisplatin, gemcitabine, taxol and a mixture of cisplatin and gemcitabine) appear in Table 1.

TABLE 1
in vitro cytotoxicity data for a 72 h incubation of the compounds with the ovarian
cancer cell lines (A2780and A2780cisR (left) and the in vivo efficacy study of
cisPt(Gem)(PhB) on the Lewis lung cancer murine model.

| | | | In vivo efficacy LLC | | |
|---|---|---|---|---|---|
| | In vitro IC₅₀ (μM) | | | | Inhibition of |
| | Cancer cell lines | | Daily dose (mg | tumor weight | tumor growth |
| Compound | A2780 | A2780cisR | kg⁻¹) | (g) | (%) |
| ctc-[Pt(NH₃)₂(Tax- | 0.004 ± 0.002 | 0.0059 ± 0.00004 | ND^{a} | ND^{a} | ND^{a} |
| Carb)(OH)Cl₂] | | | | | |
| Taxol | 0.0022 ± 0.001 | 0.003 ± 0.00006 | NDa | ND | ND^{a} |
| ctc-[Pt(NH₃)₂(Gem- | 0.063 ± 0.01 | 0.066 ± 0.002 | ND^{a} | ND^{a} | ND^{a} |
| Suc)(PhB)Cl₂] | | | | | |
| ctc-[Pt(NH₃)₂(Gem- | 0.005 ± 0.0005 | 0.014 ± 0.001 | 20 | 0.032 ± 0.003 | 92.2 |
| Carb)(PhB)Cl₂] | | | | | |
| Cisplatin | 0.924 ± 0.05 | 16.25 ± 3.14 | 3 | 0.040 ± 0.01 | 90.3 |
| Gemcitabine | 0.002 ± 0.001 | 0.005 ± 0.001 | 60 | 0.08 ± 0.2 | 80.5 |
| Cisplatin + | 0.0031 ± 0.0002 | 0.0121 ± 0.0001 | 60 + 3 | 0.058 ± 0.03 | 85.9 |
| Gemcitabine (1:1) | | | | | |
| Control | | | none | 0.41 ± 0.1 | |

^{a}ND = Not determined

Taxol is much more potent than cisplatin and ctc-[Pt(NH₃)₂(Tax-Carb)(OH)Cl₂] is only slightly less potent than taxol. Gemcitabine is significantly more cytotoxic than cisplatin and also somewhat more potent than ctc-[Pt(NH₃)₂(Gem-Carb)(PhB)Cl₂] and ctc-[Pt(NH₃)₂(Gem-Suc)(PhB)Cl₂]. The triple-action ctc-[Pt(NH₃)₂(Gem-Carb)(PhB) Cl₂] and ctc-[Pt(NH₃)₂(Gem-Suc)(PhB)Cl₂] have IC₅₀ values in the low nM range, significantly more potent than cisplatin. Interestingly, the nature of the linkage between the Gem and the Pt^{IV} has a large effect on cytotoxicity. ctc-[Pt(NH₃)₂(Gem-Carb)(PhB)Cl₂], with the carbonate linkages is about 12- and 5-fold more potent than its analog with the succinate bridge in A2780 and A2780cisR respectively. The reasons for these differences are currently under investigation.

In vitro cytotoxicity does not predict the ability of the compound to reach the tumor site and to kill cancer cells in the tumor mass. Moreover, it provides no information on the toxicity of the compounds. Therefore, we also carried out preliminary in vivo studies comparing cisplatin, gemcitabine, ctc-[Pt(NH₃)₂(Gem-Carb)(PhB)Cl₂] and co-treatment with cisplatin and Gemcitabine in the murine Lewis Lung Carcinoma (LLC) solid tumor model. Seven days after tumor inoculation the tumor-bearing mice were randomized into vehicle control and treatment groups. Control mice received the vehicle (0.5% DMSO (v/v) and 99.5% of a saline solution (v/v)), whereas treated groups received daily doses of ctc-[Pt(NH₃)₂(Gem-Carb)(PhB)Cl₂] (20 mg kg⁻¹ in vehicle solution, orally) gemcitabine (60 mg kg⁻¹ in 0.9% saline solution, iv) or cisplatin (3 mg kg⁻¹ in saline solution, iv). The tumor growth was evaluated at day 15, and the results are summarized in Table 2. As an estimation of the adverse side effects, changes in the body weights were monitored every two days.

TABLE 2
The in vivo antitumor activity of the triple-action Pt(IV)
prodrug ctc-[Pt(NH₃)₂(Gem-Carb)(PhB)Cl₂], Gem and
cisplatin evaluated in LLC solid tumor. Days 7-14:
animals received daily oral savage of 20 mg kg⁻¹ of ctc-
[Pt(NH₃)₂(Gem-Carb)(PhB)Cl₂], daily i.p. of 3 mg kg⁻¹ of
cisplatin, 60 mg kg⁻¹ of Gem or a combination of 3 mg kg⁻¹ of
cisplatin and 60 mg kg⁻¹ of Gem. At day 15 animals were sacrificed.

| | Daily | Average tumor | Inhibition of |
|---|---|---|---|
| | dose | weight (mean ± | tumor |
| | (mg kg⁻¹) | SD, g) | growth (%) |
| Control | | 0.41 ± 0.1 | — |
| ctc-[Pt(NH₃)₂(Gem- | 20 | 0.032 ± 0.003 | 92.2 |
| Carb)(PhB)Cl₂] | | | |
| Cisplatin | 3 | 0.040 ± 0.01 | 90.3 |
| Gem | 60 | 0.080 ± 0.2 | 80.5 |
| Gem + Cisplatin | 60 + 3 | 0.058 ± 0.03 | 85.9 |

Although most potent in vitro, Gem was the least effective in vivo and induced a reduction of tumor growth of about 80%. Noteworthy, oral administration of ctc-[Pt(NH₃)₂(Gem-Carb)(PhB)Cl₂] induced a ca. 92% reduction of the tumor mass compared to the control group, very similar to the result obtained with cisplatin (˜90% tumor inhibition). Remarkably, the time course of body weight changes indicated that treatment with ctc-[Pt(NH₃)₂(Gem-Carb)(PhB)Cl₂] induced a body weight loss lower than that induced by cisplatin. On the contrary, Gem induced a reduction of tumor growth of about 80% and a significant anorexia, with a body weight loss ˜20%. The co-treatment with cisplatin and Gemcitabine was less effective than ctc[Pt(NH₃)₂(Gem-Carb)(PhB)Cl₂] in inhibition of tumor growth (86 vs. 92%) but more importantly it was significantly more toxic than ctc-[Pt(NH₃)₂(Gem-Carb)(PhB)Cl₂] resulting in a body weight loss >30%.

### Multi-Action Pt^{IV} Prodrugs With Combretastatin-A4 (CA-4)

The following compounds were additionally prepared and analyzed:

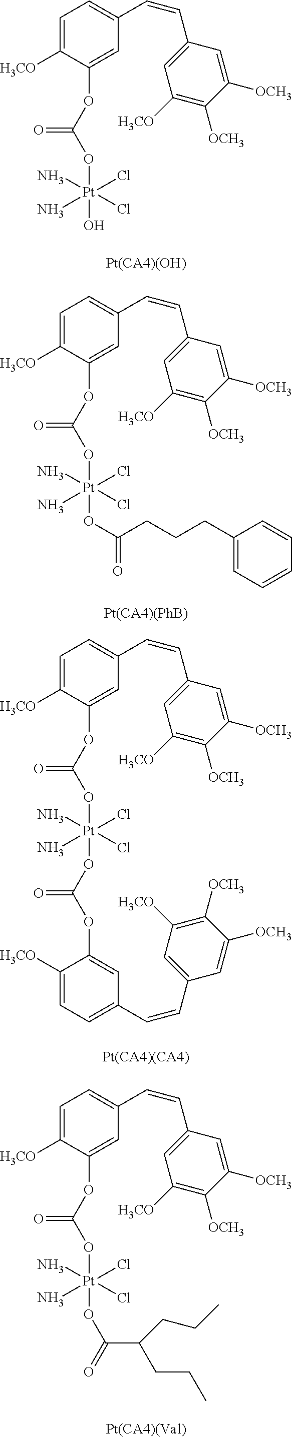

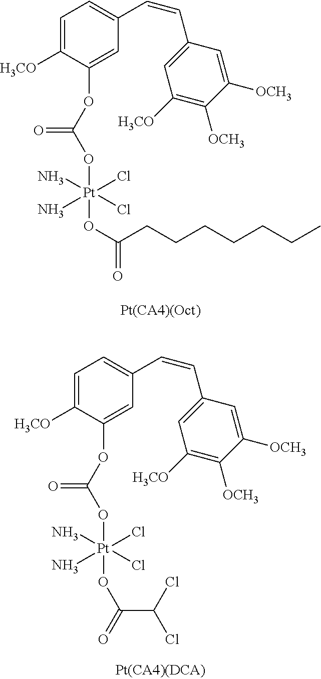

The cytotoxicity data for the above compounds is provided in Table 3.

TABLE 3
Cytotoxicity data.

| | A2780 | | A2780 res | | A375 | | PC9 | | |
|---|---|---|---|---|---|---|---|---|---|
| | mean | error | mean | error | mean | error | mean | error | Unit |
| Cisplatin | 1.56 | 1.01 | 25.56 | 6.77 | 2.74 | 2.02 | 14.40 | 7.31 | μM |
| CA4 | 2.12 | 0.06 | 2.63 | 0.23 | 1.63 | 0.61 | 4.87 | 0.40 | nM |
| Pt(CA4)(OH) | 3.28 | 0.75 | 4.74 | 0.63 | 2.91 | 1.20 | 6.36 | 1.22 | nM |
| Pt(CA4)(PHB) | 3.95 | 0.49 | 4.81 | 0.44 | 3.43 | 1.18 | 6.94 | 0.62 | nM |
| Pt(CA4)(CA4) | 2.03 | 0.41 | 2.67 | 0.79 | 1.64 | 1.08 | 4.86 | 0.84 | nM |
| Pt(CA4)(Val) | 5.37 | 1.43 | 6.19 | 1.08 | 4.41 | 2.23 | 9.98 | 0.93 | nM |
| Pt(CA4)(Oct) | 4.20 | 0.78 | 4.89 | 0.94 | 3.91 | 1.24 | 7.75 | 2.06 | nM |
| Pt(CA4)(DCA) | 3.56 | 0.15 | 4.06 | 0.06 | 2.74 | 1.22 | 6.18 | 0.59 | nM |
| Oxoplatin | 0.45 | | 1.17 | | 2.21 | | 3.99 | | μM |
| Oxp(CA4)(OH) | 9.27 | | 13.96 | | 8.11 | | 19.78 | | nM |

These results indicate that conjugating a very cytotoxic moiety (CA-4) to the Pt(IV) derivatives of cisplatin or oxaliplatin results in very potent multi-action prodrugs. Clearly the more potent CA-4 with nM IC₅₀ (compare to the μM cisplatin) determines the cytotoxicity of the prodrugs with relatively little effect of the second axial ligand. While it may seem that conjugating the CA-4 to the Pt does not results in improved cytotoxicity it should be remembered that it can significantly alter the in vivo results as the Pt(IV) prodrugs demonstartes different biodistribution, pharmacokinetics and toxicity than the CA-4 and co-treatment of CA-4 and cisplatin (see example of the in vivo study of the gemcitabine analog).

### Multi-Action Pt^{IV} Prodrugs With Taxol (Paclitaxel)

The taxol derivative of the below structure was prepared:

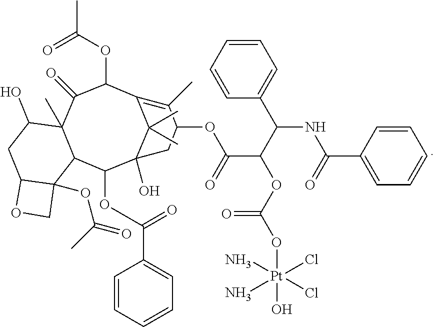

TABLE 4
Cytotoxicity data for the taxol compound of the invention.

| | A2780 | A2890cisR | A375 | PC9 |
|---|---|---|---|---|
| CisPt(Taxol)(OH) | 3.1 nM | 5.9 nM | 18 nM | 6 nM |
| Taxol | 1.05 nM | 3.1 nM | 10 nM | 4.5 nM |
| cisplatin | 1.02 μM | 20.7 μM | 1.76 μM | 3.5 μM |

In summary, we developed a new approach that allows to conjugate bioactive molecules with OH groups to the axial position of Pt^{IV} in a manner that following reduction of the Pt^{IV}, the bioactive molecule is released in its active form. We accomplished this by using a carbonate as a bridge between the hydroxido axial ligand of Pt^{IV} complexes and OH groups of the bioactive molecules. Following reduction, the carbonated ligands lose CO2 and rapidly generate the original bioactive molecules with an OH group. This allowed us to prepare multi-action Pt^{IV} derivatives of cisplatin with drugs such as gemcitabine, taxol and estramustine.

We studied the anticancer properties of ctc-[Pt(NH₃)₂(Gem-Carb)(PhB)Cl₂] and noted that in the in vitro studies it was significantly more potent than cisplatin but slightly less potent than Gemcitabine and comparable to the co-treatment with cisplatin+Gemcitabine. Importantly, in the in vivo studies ctc-[Pt(NH₃)₂(Gem-Carb)(PhB)Cl₂] was significantly more potent and less toxic than the co-treatment with cisplatin and gemcitabine demonstrating the advantage of using multi-action prodrugs as compared to combination therapy.

We believe that this novel approach, paves the way for the design and synthesis of novel classes of multi-action Pt^{IV} anticancer agents with a variety of bioactive moieties that have hydroxyl functional groups that were hitherto unavailable to the medicinal chemists.

While succinates can be used to bridge the bioactive ligands with the Pt^{IV}, we have shown that while the differences in the rates of reduction between Pt^{IV} complexes with carbonate and succinate bridges to the hydroxy groups of the ligands, are not very large, there is a huge difference in the rates of release of the active moiety, due to the slow hydrolysis of the ester bond. Nevertheless, these results may not necessarily reflect the relative rates of release of the bioactive moieties in cells.

In vivo experiments: 5-week-old female Balb/c mice will be purchased from InVivos (Singapore). Animals will be housed in animal-holding units at the National University of Singapore (NUS) in a pathogen-free environment at constant temperature in a 12/12-hour light/dark cycle. All animals procedures will be carried out according to a protocol approved by the National University of Singapore Institutional Animal Care & Use Committee, Protocol Number: R16-1204. The mice will be acclimatized for 2 weeks following arrival before testing. Mice will be allowed free access to food and water. The mice will be shaved one day before the subcutaneous injection of CT26 cells. CT26 cells (3×10⁶ cells in 50 μd of sterile PBS) will be injected subcutaneously on the back of the mice. The injections of tested compounds will start when tumors become palpable. Subsequently, animals will be separated into several groups (5 mice in each group) and will be injected (either intravenously via tail vein or intraperitoneally) with the respective drug and Pt(IV) complexes in sterile PBS on days 10, 17 and 24. Control groups will be injected with 200 μL of sterile PBS without the drug Animals will be controlled for distress development. Their weight changes will be monitored every other day for 40 consecutive days.

Encapsulation in drug delivery systems: Pt(IV) complexes will be encapsulated inside synthetic (e.g. liposomes or micelles) and/or cell-derived nanovesicles. In vitro stability studies and drug release profiles will be evaluated through membrane dialysis and quantified by HPLC.

The injection volume for the nanoformulations will be calculated based on the drug encapsulation efficiency (EE %), to yield the final concentration of Pt 1.95 mg/kg.

Evaluation of efficacy: Tumour size will be measured by calliper and tumour volume will be calculated using the formula Volume =(length×width²)/2. Efficacy will be expressed as tumor volume against time, over a period of up to 4-5 weeks. Once the diameter of the tumor exceedes 150 mm³ or upon development of ulceration, the mice will be immediately euthanized.

Organ distribution studies: CT26 cells (3×10⁵ cells in 50 μl of PBS) will be injected subcutaneously on the back of the mice. Once the tumors become palpable and reach around 50 mm³, the mice (4-5 mice in each group) will be injected with compounds of interest and euthanized 24 h later. The heart will be re-perfused with 30 ml of physiological saline immediately before the excision of the organs. The brain, liver, kidney, spleen, intestine, heart, lung and tumor tissue will be collected from each mouse and flash-frozen in liquid nitrogen. The organs of the blank mouse will be used for the measurements of the background. The organs will be lyophilized for 4 d, weighed, mechanically minced and digested in 500 μL of ultrapure 65% HNO₃ at 100° C. for 3 d. The resulting solution will be diluted to 2% v/v HNO₃ with ultrapure Milli-Q water. Platinum content will be measured by ICP-MS as described above.

Statistical analysis: Statistical analysis will be performed by two-tailed t-test and two-way ANOVA with Bonferroni post-hoc analysis using GraphPad Prism software (GraphPad Software Inc., CA) with p<0.05 considered as significant (*p<0.05, **p<0.01, ***p<0.001, ns−p >0.05). Error bars represent standard error of the mean (SEM) of at least three independent reproducible biological experiments.

1. A method of conjugating a hydroxido group of a Pt complex and a heteroatom of a hydroxyl-, an amine- or a thiol-containing material, the method comprising reacting the Pt complex with the hydroxyl-, an amine- or a thiol-containing material, under conditions causing association between the hydroxide group and the heteroatom, wherein (a) the oxygen atom of the hydroxido group or (b) the oxygen atom of the hydroxyl-containing material, or a nitrogen atom of the amine-containing material, or a sulfur atom of the thiol-containing group is activated.

2. The method according to claim 1, for conjugating the hydroxido group in a Pt complex and an oxygen atom of a hydroxyl-containing material, the method comprising reacting the Pt complex with the hydroxyl-containing material, wherein (a) the oxygen atom of the hydroxido group or (b) the oxygen atom of the hydroxyl-containing material is activated.

3. The method according to claim 1, for conjugating the hydroxido group in a Pt complex and a nitrogen atom of an amine-containing material, the method comprising reacting the Pt complex with the amine-containing material, wherein (a) the oxygen atom of the hydroxido group or (b) the nitrogen atom of the amine-containing material is activated.

4. The method according to claim 1, for conjugating the hydroxido group in a Pt complex and a sulfur atom of a thiol-containing material, the method comprising reacting the Pt complex with the thiol-containing material, wherein (a) the oxygen atom of the hydroxido group or (b) the sulfur atom of the thiol-containing material is activated.

5. The method according to claim 1, wherein the Pt complex is of the structure L-Pt—(OH)m, wherein L designates the presence of four planar ligand groups and 1 axial ligand group associated with the Pt atom, OH is an axially oriented hydroxido group and m is 1 or 2, such that where m is 1, the axial ligand group L is different form OH and where m is 2, the axial ligand group is OH.

6. The method according to claim 5, wherein the Pt complex is of the general structure (IA) or (IB) below: 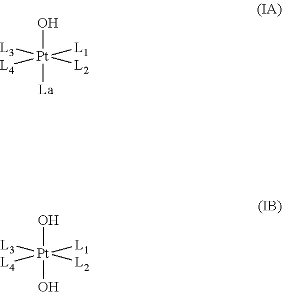
wherein each of L₁, L₂, L₃ and L₄ are same or different planar ligand groups and La is an axially oriented ligand group, being optionally different from OH.

7. The method according to claim 1 for the preparation of a compound of formula (IIA) or (IIB): 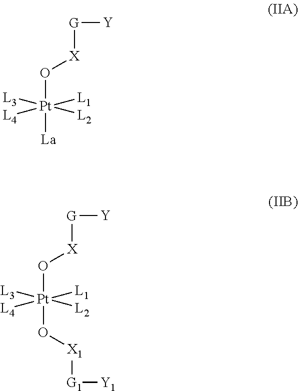
wherein
X is a linker atom or a linker group,
each of Y and Y₁, independently of the other, designates a material covalently associated to X or X₁ through a native atom G or G₁ present on the material,
each of G and G1, independently of the other, is selected from O, N, NH and S.

8. The method according to claim 7, wherein in a compound of the general formula (IIB), -X₁-G₁-Y₁ is the same as -X-G-Y.

9. The method according to claim 7, in a compound of the general formula (IIB), -X₁-G₁-Y₁ is different from -X-G-Y.

10. The method according to claim 7, wherein each of G and G1 is oxygen.

11. The method according to claim 7, wherein each of G and G1 is sulfur.

12. The method according to claim 7, wherein each of G and G1 is N or NH.

13. The method according to claim 7, wherein G is O and G₁ is S or N or NH.

14. The method according to claim 7, wherein G is N or NH and G₁ is O or S.

15. The method according to claim 7, wherein G-Y and G₁-Y₁ are each an hydroxyl-containing material.

16. The method according to claim 7, wherein G-Y and G₁-Y₁ are each an amine-containing material.

17. The method according to claim 7, wherein G-Y and G₁-Y₁ are each a thiol-containing material.

18. The method according to claim 1, wherein (a) the oxygen atom of the hydroxido group or (b) the oxygen atom of the hydroxyl-containing material, or the nitrogen atom of the amine-containing material, or the sulfur atom of the thiol-containing group is activated by a reaction with an activating agent selected from phosgene, triphosgene, bisimidazolecarbonyl or with

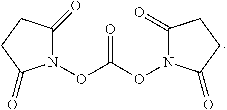 [SMILES: O=C(ON1C(=O)CCC1=O)ON1C(=O)CCC1=O]

19. The method according to claim 1, for the preparation of a compound selected from compounds having the structure: 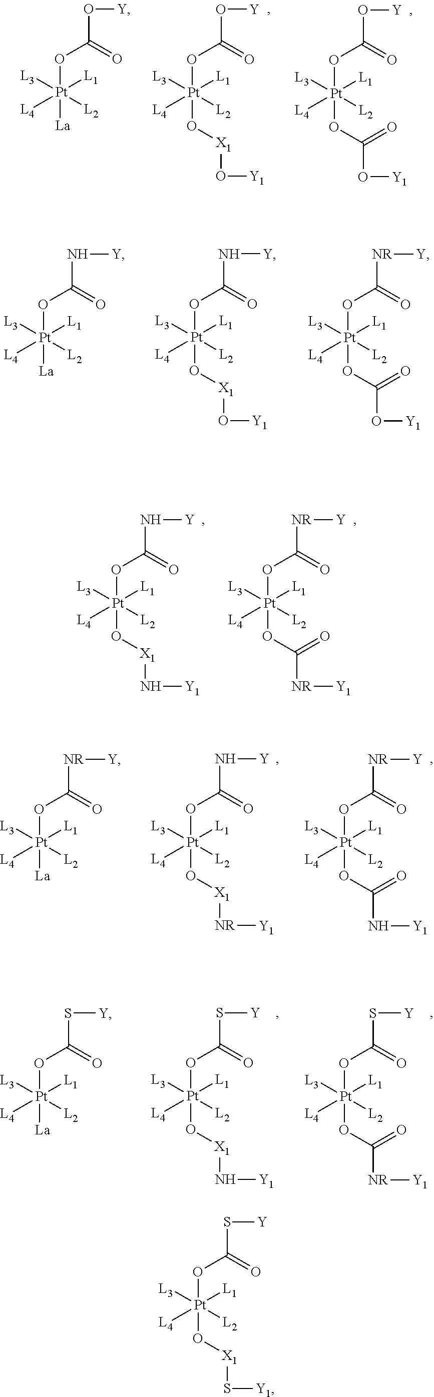 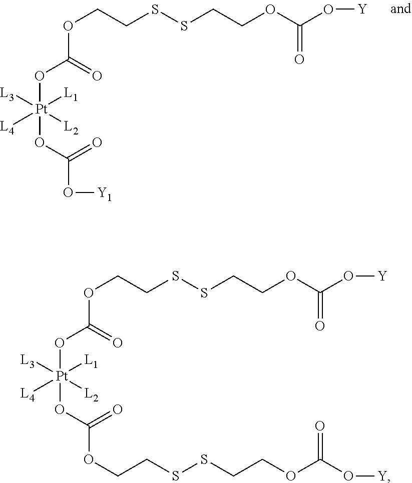
wherein
X is a linker atom or a linker group,
each of Y and Y₁, independently of the other, designates a material covalently associated to X or Xi through a native atom G or G₁ present on the material,
each of G and G1, independently of the other, is selected from O, N, NH and S.

20. A compound having the structure: 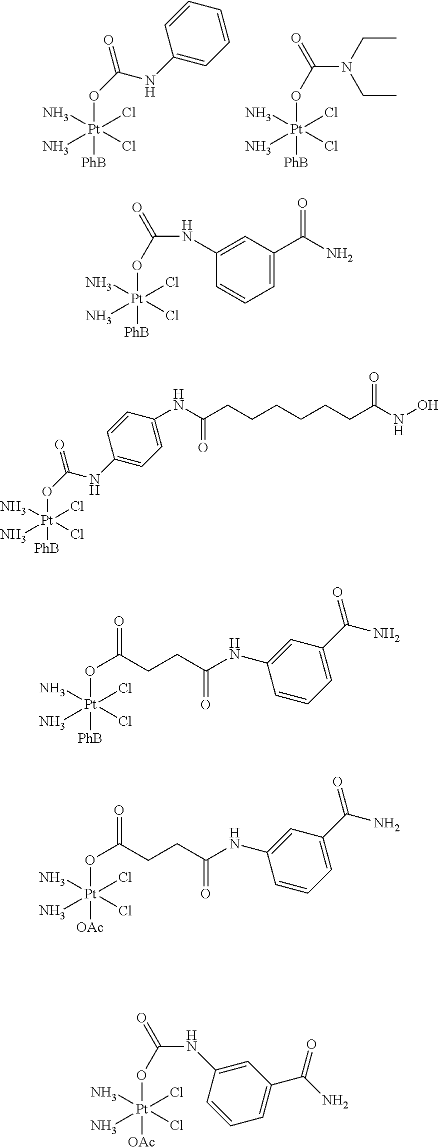
wherein in each of the structures, PhB designates phenylbutyrate and OAc designates an O-acetate group, 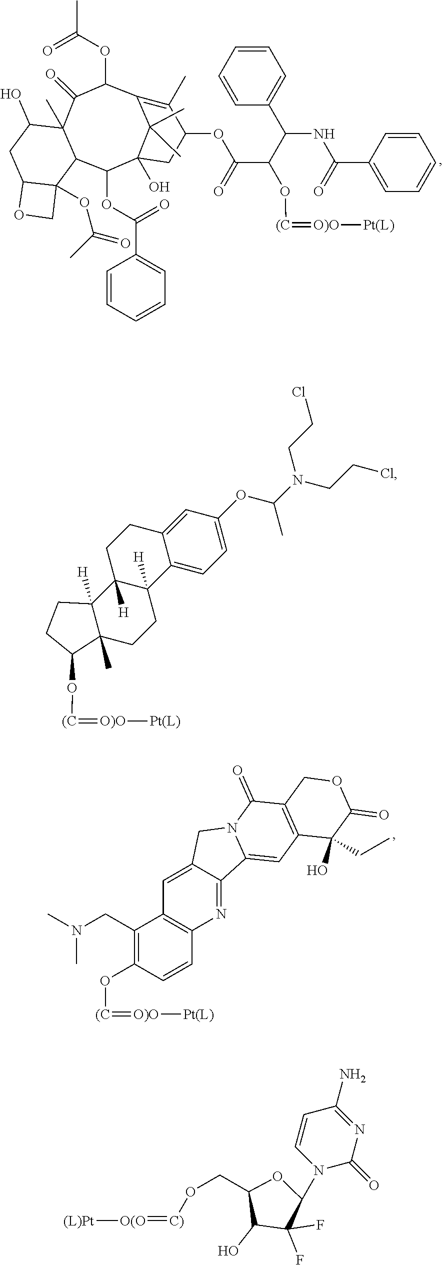 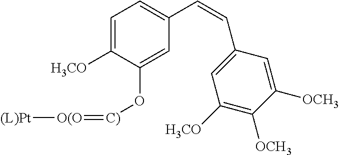
wherein Pt(L) designates a Pt complex, 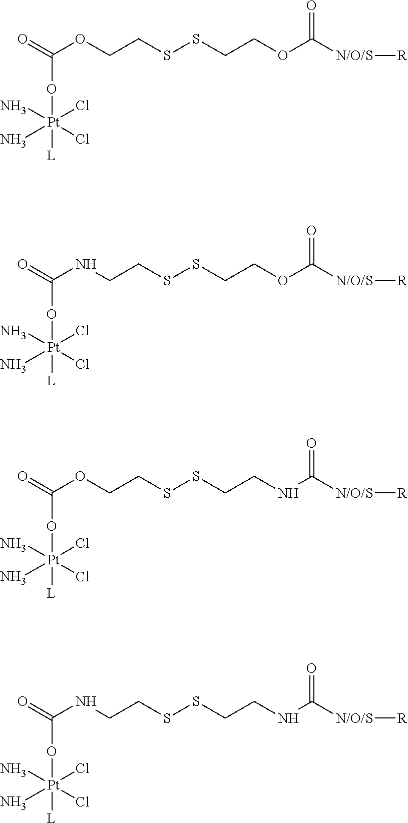 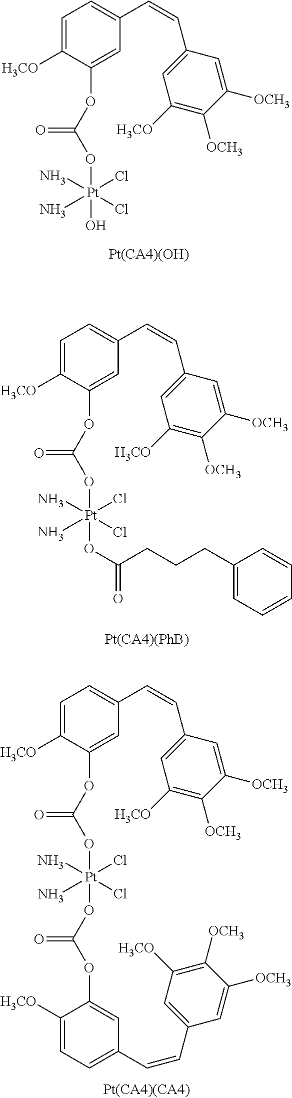 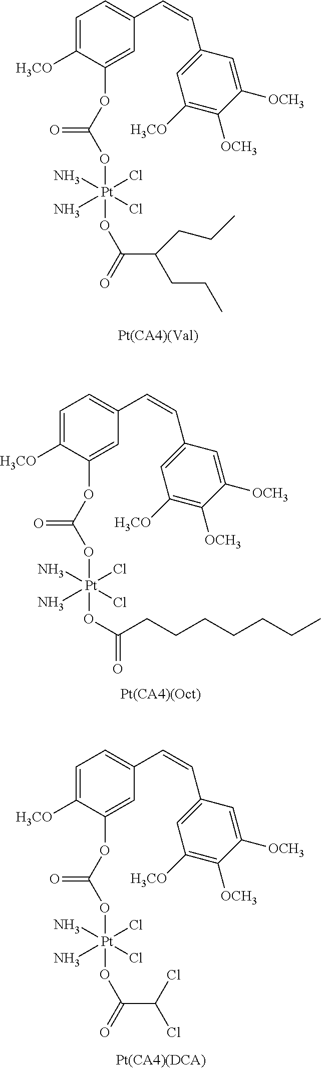 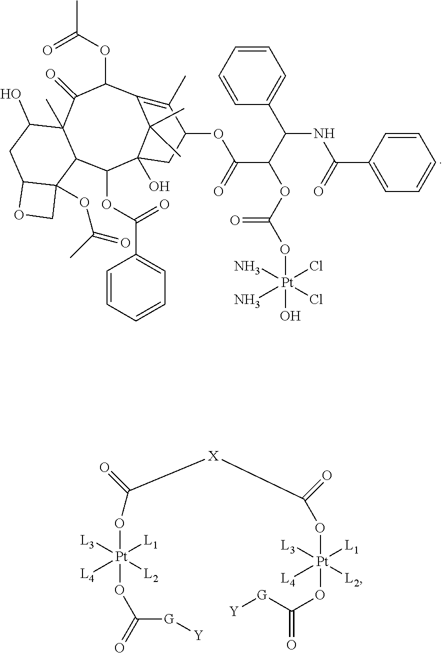
wherein
X is a linker atom or a linker group,
each of Y and Y₁, independently of the other, designates a material covalently associated to X or Xi through a native atom G or G₁ present on the material,
each of G and G1, independently of the other, is selected from O, N, NH and S, 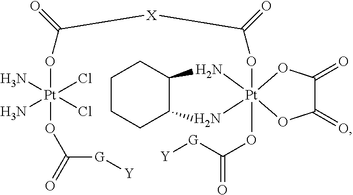
wherein
X is a linker atom or a linker group,
each of Y and Y₁, independently of the other, designates a material covalently associated to X or X₁ through a native atom G or G₁ present on the material,
each of G and G1, independently of the other, is selected from O, N, NH and S.

21. (canceled)
